(19) United States
(12) Patent Application Publication
(10) Pub. No.: US 2019/0143135 A1
(43) Pub. Date: May 16, 2019

(54) METHODS FOR TREATING GLIOBLASTOMA OR RECURRENT GLIOBLASTOMA UTILIZING A WIRELESS SIGNAL ALONE OR IN COMBINATION WITH ONE OR MORE CANCER DRUGS, AND ASSOCIATED SYSTEMS, APPARATUSES, AND DEVICES

(71) Applicant: Nativis, Inc., Seattle, WA (US)

(72) Inventor: Bennett Michael Butters, Lacey, WA (US)

(21) Appl. No.: 16/151,235

(22) Filed: Oct. 3, 2018

Related U.S. Application Data
(60) Provisional application No. 62/568,284, filed on Oct. 4, 2017.
(60) Provisional application No. 62/568,287, filed on Oct. 4, 2017.

(51) Int. Cl. A61N 1/40; A61K 39/395; A61K 31/495; C12N 15/113; A61K 31/337; A61P 35/00; A61N 1/32; A61N 1/36

(52) U.S. Cl. CPC A61N 1/40; A61K 39/3955; A61K 31/495; C12N 15/113; C12N 2310/14; A61P 35/00; A61N 1/323; A61N 1/36002; A61K 31/337

(57) ABSTRACT

Disclosed herein are methods and systems for treating cancer including glioblastoma, recurrent glioblastoma, or newly diagnosed glioblastoma, using the administration of ultra-low radio frequency energy (ulRFE®), either alone or in combination with one or more conventional cancer therapies. In some embodiments, the one or more conventional cancer therapies include chemotherapy or an anti-angiogenic therapy or other therapies.

### PRIORITY CLAIM

This application claims priority to U.S. Provisional Patent Application No. 62/568,284, filed on Oct. 4, 2017, and U.S. Provisional Patent Application No. 62/568,287, filed on Oct. 4, 2017, the entire contents of which are incorporated herein by reference and relied upon.

### BACKGROUND

Radio frequency energy (RFE) exposure in the 3 kHz to 3,000 GHz range has a measurable effect on human cells, and electromagnetic (EM) radiation in the RF range can impact cellular function in vitro and in vivo, without tissue heating. The magnetic field component of radiofrequency waves on living cells is likely a direct mechanism, as even weak magnetic fields affect cell function. The hypothesis that molecular interaction has a stronger EM component than previously thought is supported by computational evidence. In addition, molecules in solution generate a weak magnetic field as they stretch, twist, tumble and vibrate in an aqueous medium, and these magnetic fields are exceptionally weak, in the order of femto-Tesla (fT) in strength. These magnetic fields (as well as the electrostatic charge on the molecules) may be critical for molecular recognition and non-covalent binding in many biological processes.

Cancer, i.e., malignant neoplasm, includes a broad group of diseases that involve unregulated cell growth. In 2007, cancer attributed to approximately 13% of all human deaths worldwide, approximately 7.9 million people. Traditional treatments for cancer, such as chemotherapy, radiation therapy, and surgery, can be intrusive, life altering, and leave the patient unable to perform routine day-to-day functions. Although targets and treatments have been identified for cancer therapies, the issue of delivery remains an obstacle to overcome. Glioblastoma (GBM) is the most common primary intracranial neoplasm and the most malignant form of astrocytomas. The incidence of GBM increases steadily above forty-five years of age with a prevalence of approximately 7500 cases annually in the United States. Despite numerous attempts to improve the outcome of patients with GBM, the 5-year survival of these patients is only 10%, with median survival of 14 months. Essentially all patients will experience disease recurrence. For patients with recurrent disease, conventional chemotherapy is generally ineffective, with response rates less than 20%. With dismal prognoses and few effective treatments, new therapies are critically needed for brain cancer patients.

### SUMMARY

Provided herein in some embodiments are methods for treating cancer by administering ultra-low radio frequency energy (ulRFE®) either alone or in combination with one or more conventional cancer therapies. In some of these embodiments, the one or more conventional cancer therapies are administered before, during, or after administration of ulRFE®. In some embodiments, the subject can be simultaneously treated with ulRFE® and one or more conventional cancer therapies. In some of these embodiments, ulRFE® is administered using the Nativis Voyager® system, and in some of these embodiments the system utilizes a single signal. In some embodiments, the cancer is glioblastoma (GBM), such as recurrent glioblastoma (rGBM) or newly diagnosed GBM. In some embodiments, the one or more conventional cancer therapies include chemotherapy and/or an anti-angiogenic therapy, e.g., Avastin®.

Provided herein in some embodiments is the use of the Nativis Voyager® system to administer ulRFE® to a subject with cancer. In some embodiments, the subject is also treated with one or more conventional cancer therapies. In some of these embodiments, the system utilizes a single ulRFE® signal. In some of these embodiments, the one or more conventional cancer therapies are administered before, during, or after administration of ulRFE®, and in some embodiments, the subject is simultaneously treated with ulRFE® and the one or more conventional cancer therapies. In some embodiments, the cancer is GBM, such as rGBM or newly diagnosed GBM. In some embodiments, the one or more conventional cancer therapies include chemotherapy and/or an anti-angiogenic therapy, e.g., Avastin®.

Also provided herein in some embodiments are methods for treating cancer by administering ulRFE® to a subject with cancer. In some of these embodiments, ulRFE® is administered using the Nativis Voyager® system, and in some of these embodiments, the system utilizes a single signal derived from a single molecule. In other embodiments, the system utilizes two or more signals, each signal derived from a different molecule. In some of the other embodiments, one or more of the signals are derived from the same molecule. In some embodiments, the system utilizes three or more signals derived from three or more different molecules, e.g., three signals, four signals, five signals, or more. In some embodiments, the cancer is GBM, such as rGBM or newly diagnosed GBM.

Further provided herein in some embodiments is the use of the Nativis Voyager® system to administer ulRFE® to a subject with cancer. In some of these embodiments, the system utilizes a single signal derived from a single molecule, two signals derived from two molecules, or three or more signals derived from three or more molecules. In some of these embodiments, one or more of the two, three, or more signals are derived from the same molecules. In other embodiments, one or more of the two, three, or more signals are derived from different molecules. In some embodiments, the cancer is GBM, such as rGBM or newly diagnosed GBM.

### BRIEF DESCRIPTION OF THE DRAWINGS

Many aspects of the present technology can be better understood with reference to the following drawings. The components in the drawings are not necessarily to scale. Instead, emphasis is placed on illustrating clearly the principles of the present technology. Accordingly, various elements may be arbitrarily enlarged to improve legibility. For ease of reference, throughout this disclosure identical reference numbers may be used to identify identical or at least generally similar or analogous components or features.

FIG. 1 is a diagram of a system in use on a canine patient;

FIG. 2 is another diagram of the system of FIG. 1;

FIG. 3 is a diagram of variations of coils used for providing electromagnetic or magnetic field treatment;

FIG. 4 is a diagram of variations of shapes and sizes of coils used for providing electromagnetic or magnetic field treatment;

FIGS. 5A-5B are views of the manufacture of a cable for the system;

FIG. 6 is a view of a connector for the cable;

FIG. 7 is a schematic view of the connector for the cable;

FIG. 8 is a flow diagram of a method of manufacturing a coil for the system;

FIG. 9 is an exploded view of a housing of a controller for the system;

FIGS. 10A-10E are electrical schematics of microprocessor circuitry for the controller;

FIG. 11 is an electrical schematic of memory for the controller;

FIG. 12 is an electrical schematic of various components for the controller;

FIG. 13 is an electrical schematic of an LCD interface for the controller;

FIGS. 14A-14C are electrical schematics of cognate generator circuitry for the controller;

FIGS. 15A-15B are electrical schematics of power regulation circuitry for the controller;

FIG. 16 is flow diagram of a method of operating the system;

FIGS. 17A-17B show diagrams of an example apparatus for securing the therapy system to the cranium of a human patient.

FIG. 18 is a representative graph showing a relationship between survival of a subject and a tumor response in the subject administered ulRFE® or ulRFE® in combination with the Best Standard of Care (BSC); and

FIG. 19 is representative graph depicting the relationship between survival and tumor response in a subject administered ulRFE®.

### DETAILED DESCRIPTION

The methods, apparatuses, devices, and systems described herein illustrate several embodiments of an ultra-low radio frequency energy (ulRFE®) technology-based delivery mechanism for treating cancer, e.g., GBM such as newly diagnosed GBM or rGBM.

As set forth in the examples herein, a ulRFE® signal generated using the Nativis Voyager® system was administered either alone or in combination with a chemotherapy or an anti-angiogenic therapy to a group of subjects with rGBM. Over a six-month treatment period, multiple subjects exhibited positive responses to the treatment with no significant toxicity.

Based on these results, provided herein in some embodiments are methods of treating cancer in a subject in need thereof comprising administering to the subject a ulRFE® signal, either alone or in combination with one or more conventional cancer therapies, e.g., chemotherapies or anti-angiogenic therapies. Also provided herein is the use of a system capable of generating a ulRFE® signal to administer ulRFE® either alone or in combination with administration of one or more conventional cancer therapies, e.g., chemotherapies or anti-angiogenic therapies, to a subject with cancer. In some embodiments, the system uses a signal derived from a single molecule. In some embodiments, the system uses two signals derived from two different molecules. In some embodiments, the system uses three or more signals derived from three or more different molecules. Devices, systems, apparatuses, and kits for carrying out the disclosed methods and uses are also provided.

The terms below generally have the following definitions unless indicated otherwise. Such definitions, although brief, will help those skilled in the relevant art to more fully appreciate aspects of the invention based on the detailed description provided herein. Other definitions are provided above. Such definitions are further defined by the description of the invention as a whole (including the claims) and not simply by such definitions.

“Ultra-low radio frequency energy” or “ulRFE®” refers to magnetic fields having frequencies in the range of approximately 1 Hz (or less) to 22 kHz.

“Cognate” refers to a ulRFE® containing a record of the electromagnetic properties of a molecule, including, without limitation, molecules that are therapeutic compounds, such as, siRNA, nucleic acids, proteins, or chemicals.

“Magnetic shielding” refers to shielding that decreases, inhibits or prevents passage of magnetic flux as a result of the magnetic permeability of the shielding material.

“Electromagnetic shielding” refers to, e.g., standard Faraday electromagnetic shielding, or other methods to reduce passage of electromagnetic radiation.

“Faraday cage” refers to an electromagnetic shielding configuration that provides an electrical path to ground for unwanted electromagnetic radiation, thereby quieting an electromagnetic environment.

“Time-domain signal” or “time-series signal” refers to a signal with transient signal properties that change over time.

“Sample-source radiation” refers to magnetic flux or electromagnetic flux emissions resulting from molecular motion of a sample, such as the motions of larger molecular groupings like proteins, and the effects these motions have on surface charge. Because sample source radiation may be produced in the presence of an injected magnetic-field stimulus, it may also be referred to as “sample source radiation superimposed on injected magnetic field stimulus.”

“Stimulus magnetic field” or “magnetic-field stimulus” refers to a magnetic field produced by injecting (applying) to magnetic coils surrounding a sample, one of a number of electromagnetic signals that may include (i) white noise, injected at voltage level calculated to produce a selected magnetic field at the sample of between 0 and 1 G (Gauss), (ii) a DC offset, injected at voltage level calculated to produce a selected magnetic field at the sample of between 0 and 1 G, and/or (iii) sweeps over a low-frequency range, injected successively over a sweep range between at least about 0-1 kHz, and at an injected voltage calculated to produce a selected magnetic field at the sample of between 0 and 1 G. The magnetic field produced at the sample may be readily calculated using known electromagnetic relationships, knowing a shape and number of windings in an injection coil, a voltage applied to coils, and a distance between the injection coils and the sample.

A “selected stimulus magnetic-field condition” refers to a selected voltage applied to a white noise or DC offset signal, or a selected sweep range, sweep frequency and voltage of an applied sweep stimulus magnetic field.

“White noise” refers to random noise or a signal having simultaneous multiple frequencies, e.g., white random noise or deterministic noise. Several variations of white noise and other noise may be utilized. For example, “Gaussian white noise” is white noise having a Gaussian power distribution. “Stationary Gaussian white noise” is random Gaussian white noise that has no predictable future components. “Structured noise” is white noise that may contain a logarithmic characteristic which shifts energy from one region of the spectrum to another, or it may be designed to provide a random time element while the amplitude remains constant. These two represent pink and uniform noise, as compared to truly random noise which has no predictable future component. “Uniform noise” means white noise having a rectangular distribution rather than a Gaussian distribution.

“Frequency-domain spectrum” refers to a Fourier frequency plot of a time-domain signal.

“Spectral components” refers to singular or repeating qualities within a time-domain signal that can be measured in the frequency, amplitude, and/or phase domains. Spectral components will typically refer to signals present in the frequency domain.

A “subject” as used herein is an animal, preferably a mammal. In some embodiments, the subject is a human.

A “subject in need thereof” as used herein refers to a subject who has been diagnosed with, exhibited one or more symptoms associated with, or been deemed at risk of having or developing cancer. In some embodiments, the cancer is a malignant glioma, including for example GBM, such as newly-diagnosed GBM or rGBM.

In those embodiments of the methods and uses provided herein wherein chemotherapy is administered in combination with administration of the ulRFE® signal, any chemotherapy approved for the particular type of cancer being treated can be used. Likewise, in those embodiments where an anti-angiogenic therapy is administered in combination with administration of the ulRFE® signal, any approved anti-angiogenic therapy, e.g., Avastin®, can be used.

In some embodiments of the methods and uses provided herein, ulRFE® is administered using the Nativis Voyager® system. As used herein, and described in more detail below, the terms “magnetic field,” “electromagnetic field” and similar terms are used interchangeably to describe the presentation of ulRFE® to a selected region to produce biological effects, where the presented ulRFE® has a characteristic reflecting that of a specific drug, chemical or other agent.

In some of these embodiments, the system is used to administer a ulRFE® signal obtained from a single molecule, such as a mitotic inhibitor (e.g., taxane derivatives including paclitaxel), (“AIA”) or from one or more different molecules, such as a CTLA-4 inhibitor and a PD-1 inhibitor (“A2HU”). In other words, the cognate sample for the ulRFE® signal can be a mitotic inhibitor, or the cognate samples for the ulRFE® signal can be a CTLA-4 inhibitor and a PD-1 inhibitor. In some embodiments, the inhibitor is a protein, a nucleic acid (e.g., siRNA), an inorganic compound, an organic compound, or a combination thereof. The terms “ulRFE®”, “cognate” and “signal” are at times used interchangeably herein. Some common taxane derivatives include paclitaxel, docetaxel, and cabazitaxel. Other taxane derivatives are known in the art [4, 5]. Every molecule has a specific and unique electrostatic surface potential. Electrostatic surface potential is a critically important property of a molecule; it is a key factor in how a molecule interacts with (and in) a biological system. The electrostatic surface potential of a molecule can be measured and recorded to derive a cognate using “Super Conducting Quantum Interference Device” (SQUID)-based technology. Transducing these highly precise ulRFE® profiles (cognates) into biological systems can produce precise biological responses. Transduction of these cognates induces selective charge transfer in a defined bioactive target, thus altering cell dynamics, which can produce biological effects.

The Nativis Voyager® system can produce low-level radio frequency energy (RFE) that induces a biologic response in malignant solid tumors. The encrypted RFE signal is embedded in the firmware of the Voyager controller of the system during manufacturing. For example, using RFE derived from a mitotic inhibitor, the Voyager therapy may block the division of cancer cells by blocking the disassembly of microtubule leading to aberration, multi-nucleation and disruption of mitotic spindle activity during cell division at metaphase.

In some embodiments of the methods and uses provided herein, ulRFE® and the one or more conventional therapies, e.g., chemotherapies or anti-angiogenic therapies, are administered over approximately the same time course, i.e., the first and last administrations of each occur around the same time. In other embodiments, one may be administered to the subject before the other. For example, a subject receiving chemotherapy or an anti-angiogenic therapy may have received the chemotherapy or the anti-angionenic therapy for some period prior to the first ulRFE® administration, or vice versa. Similarly, administration of one may continue after the other has ceased. For example, ulRFE® administration may continue after the last administration of chemotherapy or anti-angiogenic therapy, or vice versa.

In some embodiments of the methods and uses provided herein, ulRFE® is administered continuously during the treatment period, i.e., 24 hours/day (except for brief periods for medical procedures and personal hygiene). In other embodiments, ulRFE® is administered non-continuously, e.g., at specific intervals or at specific intervals throughout the chemotherapy or anti-angionenic therapy treatment period. In some embodiments, ulRFE® is administered in multiple cycles of the same or different lengths, e.g., multiple cycles of 4 weeks each.

Provided herein in some embodiments are methods of treating a malignant glioma, e.g., GBM, such as newly-diagnosed GBM or rGBM, in a subject in need thereof comprising administering to the subject one or more chemotherapies or anti-angiogenic therapies, and/or a ulRFE® signal, using the Nativis Voyager® system. In some embodiments, the methods of treating malignant glioma (e.g., GBM) in a subject in need thereof comprises administering a ulRFE® signal using the Nativis Voyager® system, and not one or more chemotherapies or anti-angiogenic therapies.

Use of ulRFE® may avoid problems of drug-based delivery, such as the ability of drugs to reach their intended target(s). For example, magnetic fields in the radio frequency range (derived from an alternating current (AC) source between 3 kHz to 3000 GHz) of low power and low frequency sufficiently penetrate tissue(s) [1-3], ensuring access to areas that are poorly perfused. As such, ulRFE® technologies employ signals in a frequency range of 0-22 kHz, which may modulate specific regulatory, metabolic or other pathways in humans, animals, and plants by directly regulating the production of protein, starch, sugar, fat, and other molecules in cells, or altering other cellular functions such as cell division.

Nativis Voyager® ulRFE® technology may be implemented by medical professionals and/or researchers to identify effective, safe, and less expensive alternatives to cancer therapies by developing ulRFE® transduction mechanisms for some applications. Applicant has disclosed, in related patents and patent applications noted herein, systems and methods for detecting and recording molecular cognates from chemical, biochemical, or biological molecules or from chemical, biochemical, or biological agents. In some implementations, the recordings represent molecular cognates of the chemical, biochemical, or biological molecules or agents used to provide treatment for cancer, ailments or other adverse health conditions. The methods and systems disclosed herein may be configured to deliver the effect of chemical, biochemical, or biologic treatment to a human and/or animal, without the use of drugs or chemicals, by delivering cognates derived from particular chemicals, biochemical, or biologics or their respective effects. Thus, the methods and systems allow humans and/or animals to receive an electronic exposure to electromagnetic or radio frequency energy with, for example, the click of a button. The embodiments of the systems and methods describe a system that is non-invasive, non-thermal, non-ionizing and mobile.

In some embodiments, the Voyager system comprises three components: a battery-operated controller, an electromagnetic coil, and a battery charger. In some embodiments, the electromagnetic coil is worn on the subject's head and is connected to the battery-operated controller. In some embodiments, the Voyager system provides easy and comfortable use. For example, the Voyager system can be used in a home or office environment, so that the subject can carry on with daily activities without disruption from use of the Voyager system. In some embodiments, the coil can come in a variety of sizes so that it can fit any subject's head. In other of these embodiments, a cap or headband may be worn over the coil to hide if from view or to hold it in place as needed or as desired. In some embodiments, the Voyager system does not require the subject to shave his or her head or any other special preparation for use. In some embodiments, each battery-operated controller has a battery-life of approximately 16 hours. In some of these embodiments, the subject is provided with two battery-operated controllers so that one unit may be charged using the battery charger (like a cell phone charger) while the other controller is in use. In some embodiments, recharging takes less than 2 hours, the battery-operated controller weighs only 2.7 ounces, and/or is approximately the size of a pager. In some of these embodiments, the battery-operated control is clipped to a belt or an arm band worn by the subject.

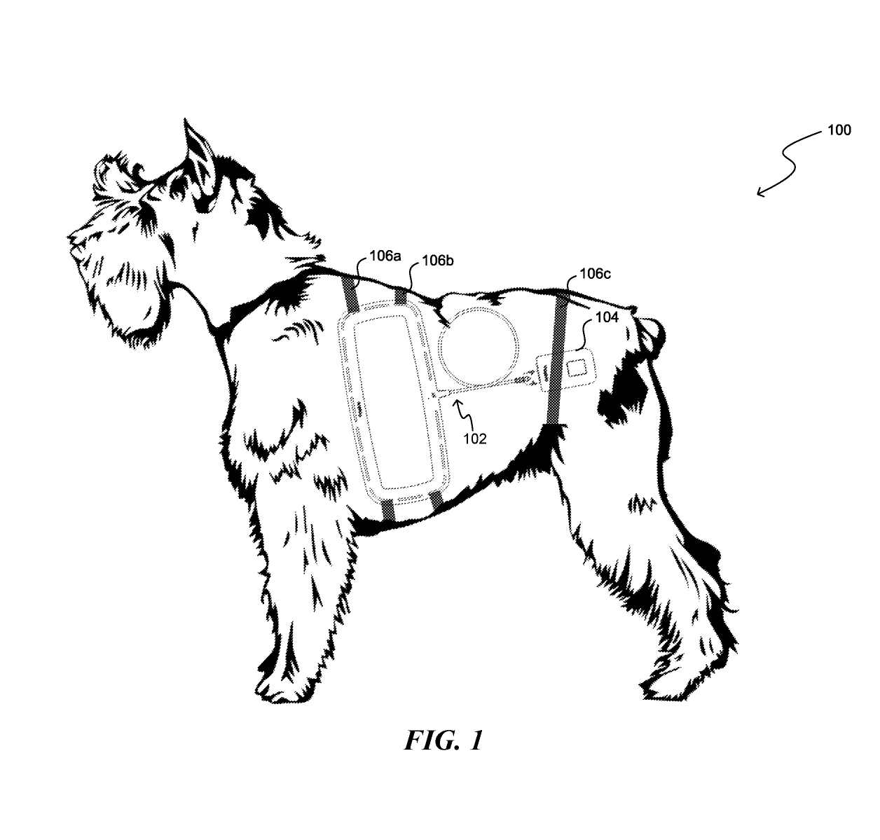

FIG. 1 illustrates an embodiment of a system 100 for applying ulRFE® cognates to an animal, such as a canine, to provide treatment, such as to selectively reduce or inhibit growth of particular types of cells. In some implementations, the system 100 may be used to treat cells by applying electromagnetic or magnetic fields to affected areas. These fields are induced or generated to expose an affected area to cognates derived from magnetic fields that emanate from drugs, chemicals or other agents. The acquisition of the cognates produced from drugs, chemicals or other agents is discussed in great detail in patent applications and patents that are co-owned by the assignees of the instant application. These patents and applications include U.S. Pat. Nos. 6,724,188, 6,995,558, 6,952,652, 7,081,747, 7,412,340, and 7,575,934; PCT Application Nos. PCT/US2009/002184, PCT/US2013/050165; and U.S. Patent Publication No. 2016/0030761A1, each of which is hereby incorporated by reference in its entirety.

The system 100 may provide various advantages over traditional treatments. For example, the system 100 may be portable and worn by humans or animals or kept near humans and/or animals as the situation requires.

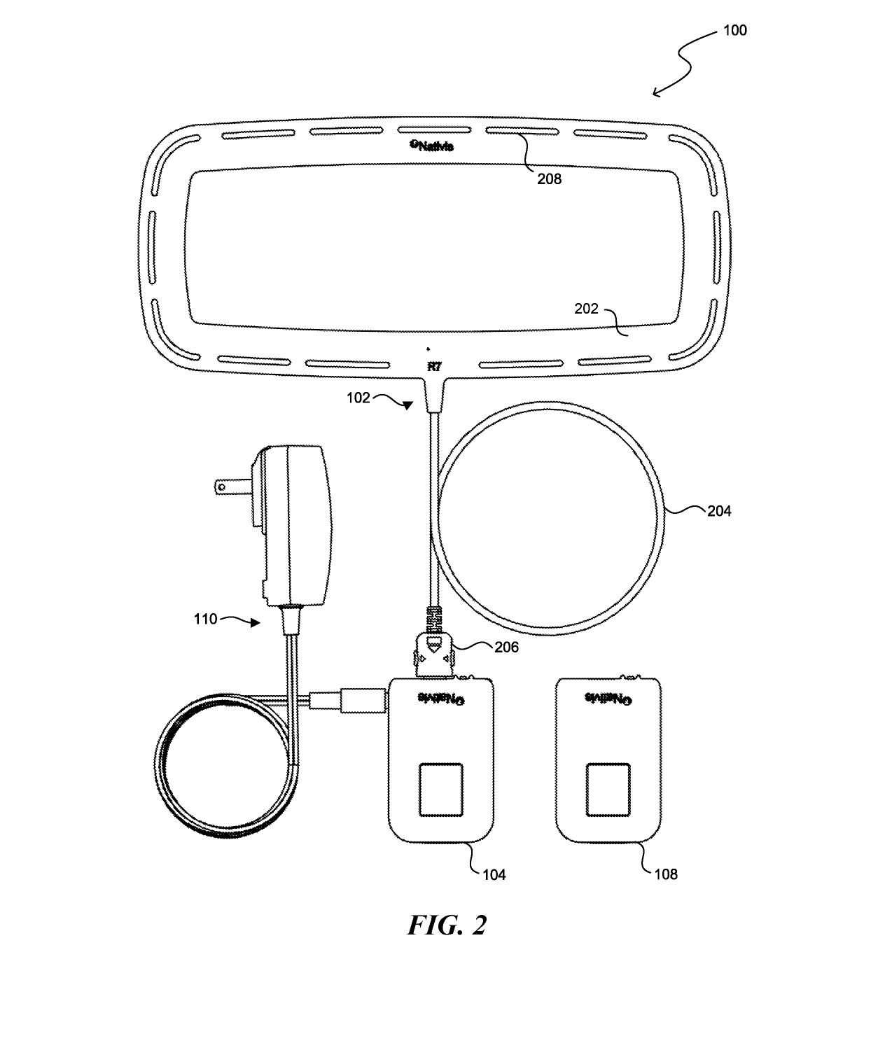

FIG. 2 illustrates the system 100 as it may be utilized. In addition to the transduction coil and cable 102 and controller 104 assemblies delivered to a user, the system 100 may also include additional coils, one or more additional controllers 108 and a battery charging device 110. For various security reasons, which are discussed below, each controller may be manufactured so that a housing for the controller cannot be opened easily.

The system 100 may also include a motion sensor (for example, accelerometer). The motion sensor can cause the controller 104 to issue an alert if sufficient undesirable motion is detected. (Of course, the motion sensor may be applied to any of the systems described above for similar purpose, such as when treatment recipient is sleeping, and moves sufficiently to cause a wearable coil 202 to potentially become dislodged, so that the alert or alarm can prompt the repositioning of the coil. The motion sensor and alarm can help ensure compliance with a treatment regime.)

In particular, the system 100 may include software stored in memory of the system (e.g., on-chip memory of a microprocessor, not shown). The software receives motion signal data from the motion sensor, which can reflect force vectors or measurements, over a period of time. The software then compares the force, direction and time of received motion data to stored rules or values to determine whether the received data represents an undesirable condition. If the system detects an undesirable condition, it can take remedial action, such as by issuing an alarm. If the system 100 includes wireless communication circuitry, the system can send an alert message to a remote monitoring facility. The system may also monitor and store global positioning information useful in determining the movement and position of livestock and field equipment.

The controller 104 can be formed of inexpensive components so as to reduce the overall cost of the system 100. Indeed, the system 100 can be configured to be disposable or of limited reusability. For example, the controller may have a system-on-chip (SoC) configuration whereby the SoC is a single semiconductor die that includes a microcontroller, memory, and analog amplifying circuitry, all monolithically formed. The controller 104 can include various types of power sources, such as a battery, capacitor, or even antenna(s) and associated circuitry so as to wirelessly obtain power that is then stored in a capacitor and used to drive the circuitry of the controller. Indeed, these and other power sources may be used for not only the system of FIG. 2, but all other systems and apparatus described herein.

### System Coil and Cable Assembly

In FIG. 2, the coil and cable assembly 102 includes an encapsulated coil 202, a cable 204, and a connector 206. The coil 202 includes one or more conductors configured to generate a magnetic or electromagnetic field from one or more cognates. As used herein, a drug or chemical-simulating cognate includes a cognate that approximately reproduces magnetic fields that emanate from one or more predetermined chemical, biochemical, and/or biological molecules or agents. The coil 202 may be configured to have various electrical characteristics. Additionally, the coil 202 may be enclosed in a plastic or other composite material to both protect the windings of the coil. As mentioned above, the system 100 may include more than one coil. Regardless of whether the system 100 is configured with one coil, or more than one coil, the coils can be flexible and malleable, can have a variety of shapes, can have different sizes or types, and can also include rigid coils. Advantageously, one or more of these coils can be externally secured to an animal to provide treatment, as opposed to subcutaneous insertion of the coil into an animal.

The controller 104 may be used in various environments. For example, the coil 202 may be placed in an animal stall, or on a bed, such as under a mattress pad of a veterinary or hospital bed or within the seat/seatback of a cart or wheelchair (or in a pillow), with the controller 104 removably attached to a frame of the cart, bed/wheelchair. As a result, a human or animal need only lie in the stall or bed to receive treatment, rather than have the coil 202 attached to the human or animal's body as described herein.

The controller 104 may store multiple cognates. The controller may then also include a software or hardware switch that allows a user to select one of the multiple cognates to be amplified and output by the controller, so that the controller may be used to generate an output of two or more cognates, such as with two matched coils (e.g., a Helmholtz coil pair), and may include two different channels, one for each of the two coils. The controller 104 can include phase control so as to control the two coils and ensure that they are in sync. Such phase control can take the form of a locking amplifier, phase lock loop circuitry, or other known means. As a result, the two coils can produce the same wireless ulRFE®, which can then be applied to a larger area.

Alternatively, the two coils can each include different geometries to account for application of the cognate to different portions of a target region, and/or to account for different geometries of the recipient. For example, if two different coils are to be positioned on a recipient's body, the coils can account for the geometries of the different locations on the body, and to account for different geometries of the target within the body (e.g., different top and side cross-sections of the same organ.)

Alternatively, rather than apply the same cognate to both coils, the controller 104 can store two or more different cognates, and apply one to coil and the other concurrently to another coil. Of course, the system 100 can include a selector that allows for both functions: applying the same cognate to both coils, or a different cognate to each of the two coils. Of course, the controller can apply two or more cognates serially, one after the other, and then loop back (e.g., apply cognates A, B and C serially in a sequence of A, B, C, A, B, . . . , though other sequences are possible). The time period for application of each cognate need not be the same, but could differ (e.g., cognate A applied for 15 minutes, B for 10 and C for 5, then the series repeats).

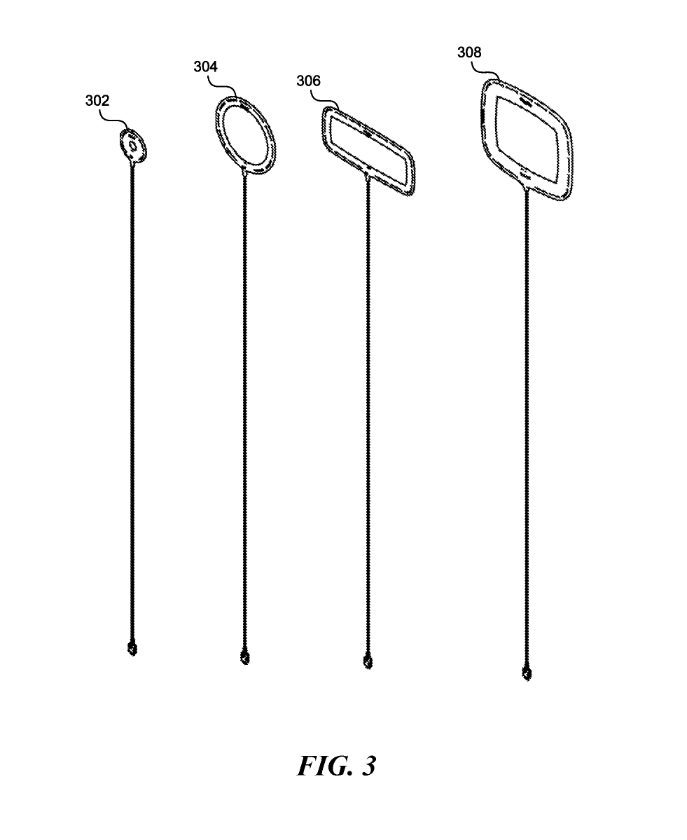

FIG. 3 illustrates diagrams of variations to the shape of the encapsulated coil 202. As illustrated, the coils used by the system 100 may include a small circular encapsulated coil 302, a large circular encapsulated coil 304, a rectangular encapsulated coil 306, a substantially square encapsulated coil 308, and/or another encapsulated coil sized and shaped to treat a particular part of the human's, mammal's, and/or animal's body. Each shape may provide advantages for treating particular parts of the body of the human, mammal, and/or animal.

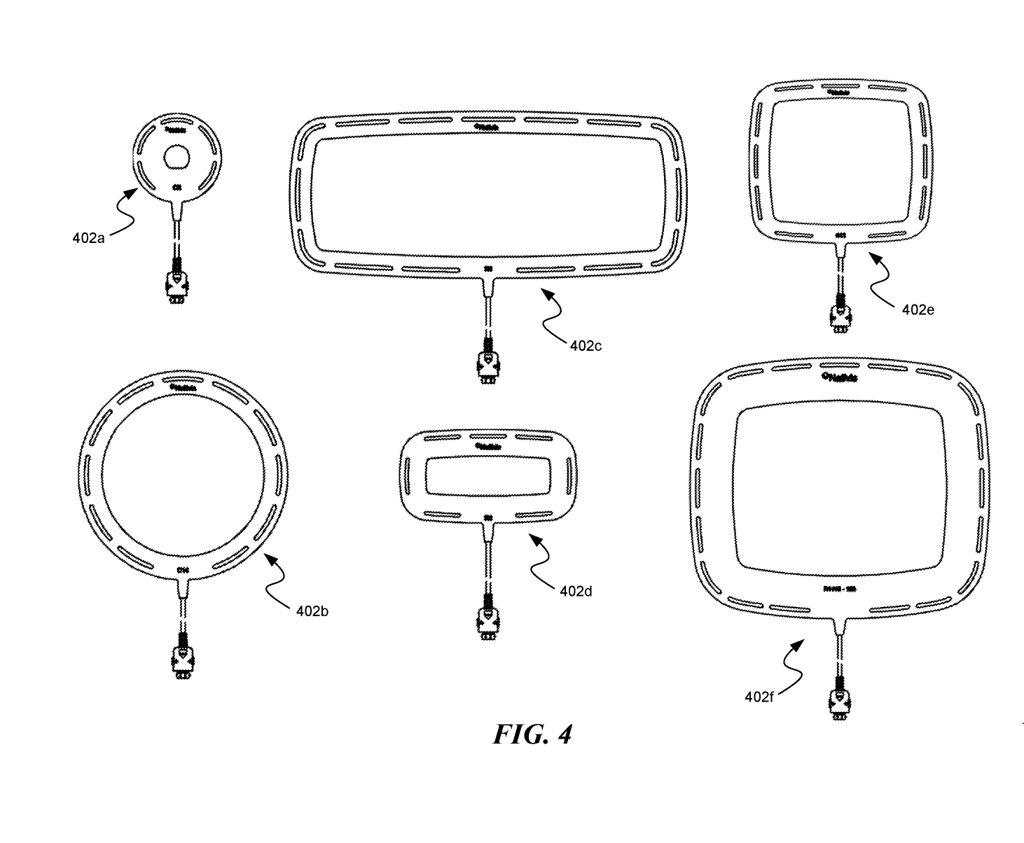

FIG. 4 illustrates examples of coils having various shapes and various dimensions. A variety of dimensions for the coils may be manufactured to more effectively apply treatment to areas that vary in size. Each of the coils 402a, 402b, 402c, 402d, 402e, and 402f can have inner and/or outer diameters or lengths, ranging from just a few centimeters to several feet, according to various implementations.

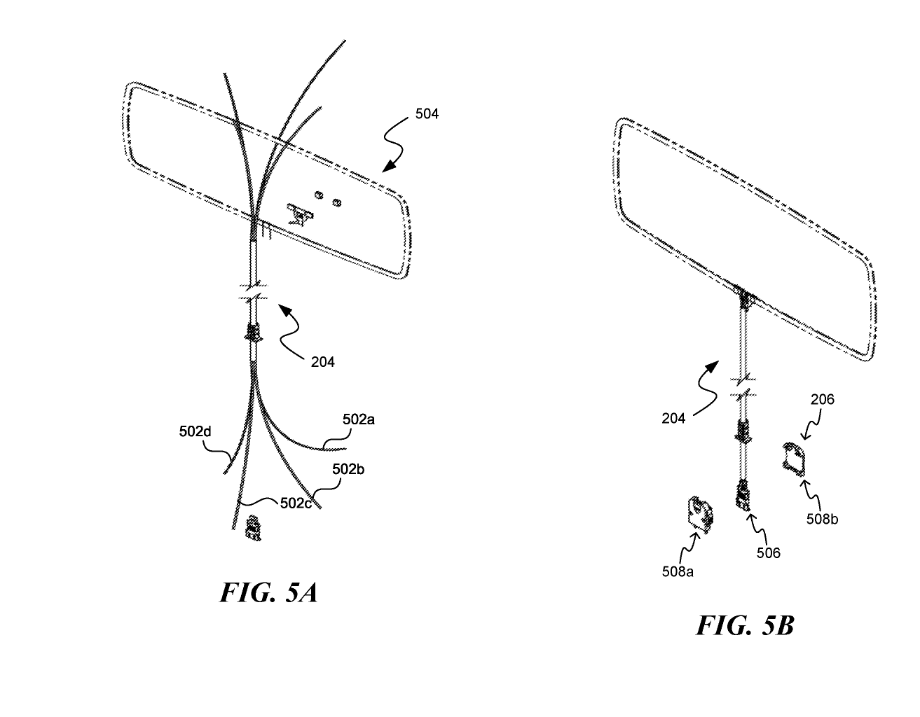

FIGS. 5A and 5B illustrate before and after diagrams of the cable 204 during manufacture. The cable 204 connects a coil, e.g., coil 202, to the connector 206 to enable the controller 104 to transmit various cognates to the coil. The cable 204 may include two or more conductors 502a, 502b, a shield 502c, and a strength-providing member 502d (collectively conductors 502). Each of the four conductors and members may be configured to perform a particular function. For example, conductors 502a and 502b may be electrically coupled to either end of the coil 504 to enable current to flow to and from the coil 504 to generate a magnetic field from the coil 504. Shield conductor 502c may be coupled to ground and be configured to provide electromagnetic shielding for the conductors 502a and 502b. Strength member 502d may be anchored to the coil 504 and to the connector 206 to provide strain relief to the conductors 502a-502c. In some implementations, the strength member 502d is manufactured with a shorter length than the other conductors so that the strength member 502d receives a majority of any strain applied between the coil 504 and the connector 206.

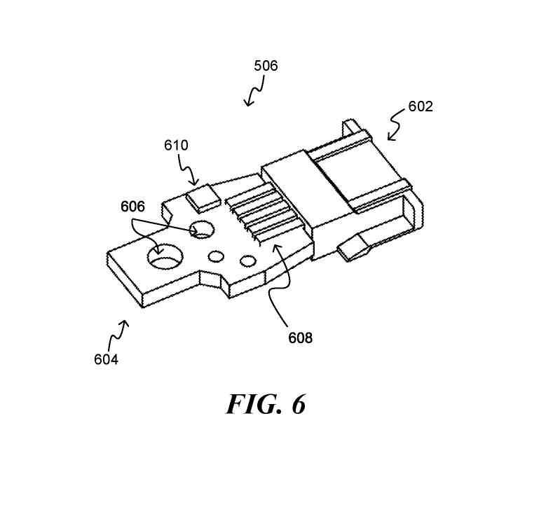

As illustrated in FIG. 5B, the connector 206 may include three parts, a connector core 506, and connector housings 508a and 508b. The connector housings 508a and 508b may encapsulate the connector core 506 to protect the traces and electronic devices carried by the connector core 506. FIG. 6 illustrates an implementation of the connector core 506. The connector core 506 has a controller end 602 and a cable end 604. The controller end 602 is configured to couple to the controller 104, and the cable end 604 is configured to provide an interface for the conductors 502. In some implementations, the strength member 502d may be anchored to one or more holes 606 to provide strain relief. The conductor core 506 may also carry a plurality of traces 608 to which the conductors 502a-c may be electrically coupled to facilitate communication with the controller 104.

As a security feature of the coil and cable assembly 102, the connector core 506 may also carry an integrated circuit 610. The integrated circuit 610 may be a microprocessor or may be a stand-alone memory device. The integrated circuit 610 may be configured to communicate with the controller 104 through the controller end 602 using communication protocols such as I2C, 1-Wire, and the like. The integrated circuit 610 may include a digital identification of the coil with which the connector core 506 is associated. The digital identification stored on the integrated circuit 610 may identify electrical characteristics of the coil, such as impedance, inductance, capacitance, and the like. The integrated circuit 610 may also be configured to store and provide additional information such as the length of the conductor of the coil, physical dimensions of the coil, and number of turns of the coil. In some implementations, the integrated circuit 610 includes information to prevent theft or reuse in a knock-off system, such as a unique identifier, cryptographic data, encrypted information, etc. For example, the information on the integrated circuit 610 may include a cryptographic identifier that represents measurable characteristics of the coil and/or the identification of the integrated circuit. If the cryptographic identifier is merely copied and saved onto another integrated circuit, for example, by an unauthorized manufacturer of the coil and cable assembly, the controller 104 may recognize that the cryptographic identifier is illegitimate and may inhibit cognate transmissions. In some implementations, the integrated circuit stores one or more encryption keys, digital signatures, stenographic data or other information to enable communications and/or security features associated with public key infrastructure, digital copy protection schemes, etc.

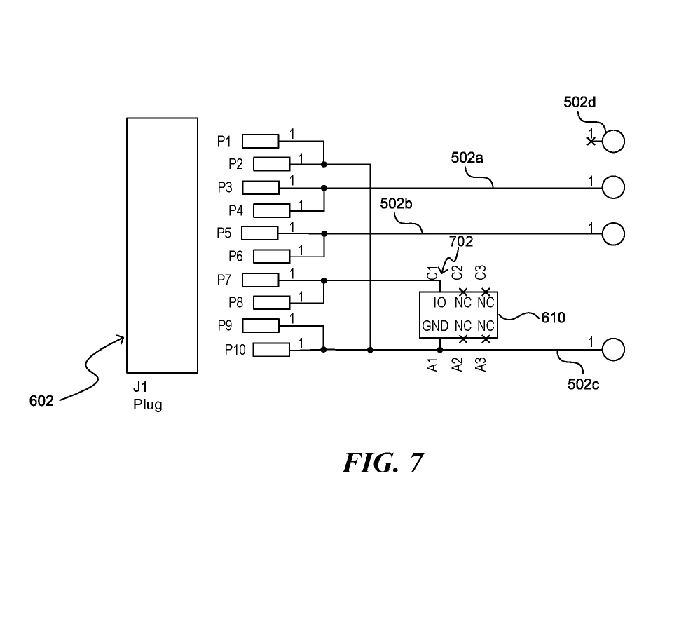

FIG. 7 illustrates a schematic diagram of the connector core 506. As shown, according to some implementations, the integrated circuit 610 may be configured to communicate with the controller 104 over a single wire, e.g., from input-output-pin 702.

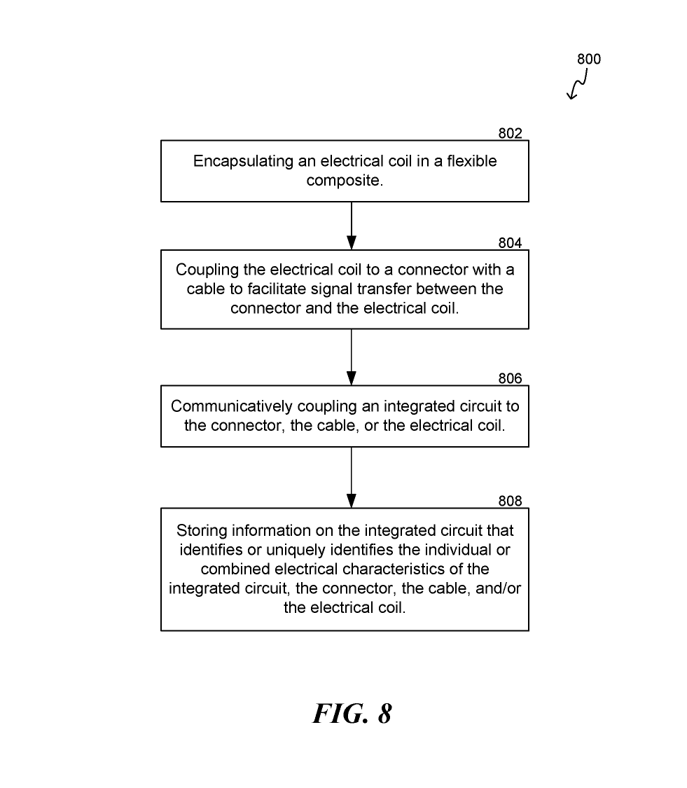

FIG. 8 illustrates a method 800 of manufacturing a coil and cable assembly, e.g., the coil and cable assembly 102, for use in providing a system that is non-invasive, non-thermal, non-ionizing and mobile.

At block 802, an electrical coil is encapsulated in a flexible composite. The flexible composite allows the electrical coil to be comfortably secured to, e.g., an animal to provide magnetic field treatment.

At block 804, the electric coil is coupled to a connector through a cable to facilitate secure transfer between the connector and the electrical coil. The cable may include multiple conductors that deliver signals between the connector and the electrical coil while providing mechanical strain relief to the signal carrying conductors.

At block 806, an integrated circuit is coupled to the connector, the cable, or the electrical coil. The integrated circuit may be coupled, for example, to the connector via one or more electrical conductors that may or may not also be coupled to the electrical coil.

At block 808, information is stored to the integrated circuit that identifies or uniquely identifies the individual or combined electrical characteristics of the integrated circuit, the connector, the cable, and/or the electrical coil. The information may be a hash or other cryptographically unique identifier that is based on information that can be unique to the integrated circuit and/or the remainder of the coil and cable assembly. This security feature can be used to prevent or deter unauthorized remanufacture of coil and cable assemblies that are compatible with the controller for the magnetic field delivery system. Additional security features are described herein, e.g., in connection with the operation of the controller for the system.

### System Controller

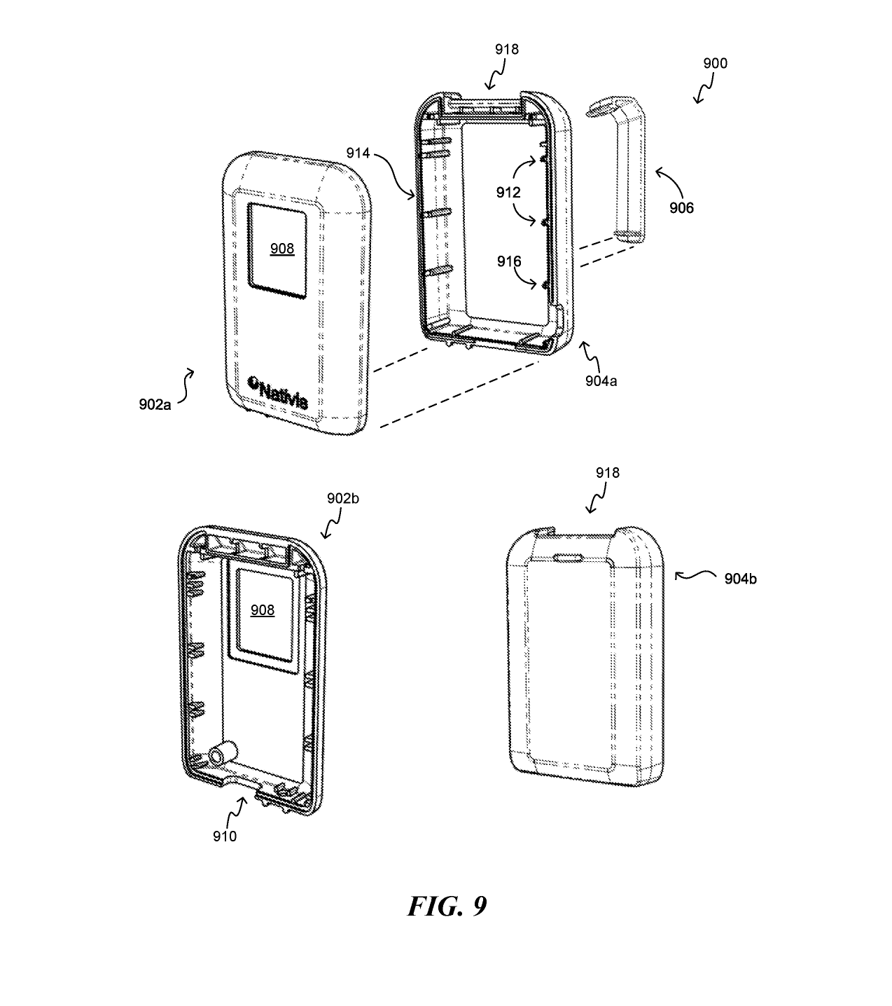

Referring briefly back to FIG. 2, the system 100 includes a controller 104 to provide an interface to the human and/or, to distribute and regulate drug and chemical-simulating cognates to the coil 202, and to prevent unauthorized copying and/or distribution of the drug or chemical-simulating cognates. According to various implementations, the controller 104 can include various features such as a housing, a processor, memory, visual and audio interfaces, in addition to other features which are described hereafter in FIGS. 9-15B.

FIG. 9 illustrates a housing 900 for the controller 104. The housing 900 may include three parts, a housing front 902 (inclusive of 902a, 902b), a housing back 904 (inclusive of 904a, 904b), and a clip 906. The housing front 902 may have a window 908 through which a visual interface may be viewed or manipulated. Although not shown, the housing front 902 may include various apertures through which buttons, dials, switches, light emitting indicators, and/or a speaker may pass or be disposed. The housing front 902 includes a cut-away or port 910 for coupling the controller 104 to the coil and cable assembly 102. The housing back 904 may include a number of pegs 912 for attaching/securing the housing back 904 to the housing front 902. While coupled together, the housing front 902 and the housing back 904 may form a seal along the edge 914, preventing water, moisture, dust, or other environmental elements from entering the housing 900. In some implementations, an adhesive or solvent is used to permanently bond the housing front 902 to the housing back 904 to deter or prevent unauthorized tampering with or viewing of the internal electronics, though in other implementations the front and back may be formed to permanently snap-fit together. As shown, the housing back 904 may include a cutout, aperture, or port 916 to allow connection to a recharging device or communication information to/from the controller 104. The clip 906 may be securely fastened or detachably coupled to slot 918 of the housing back 904 to secure or affix the controller 104.

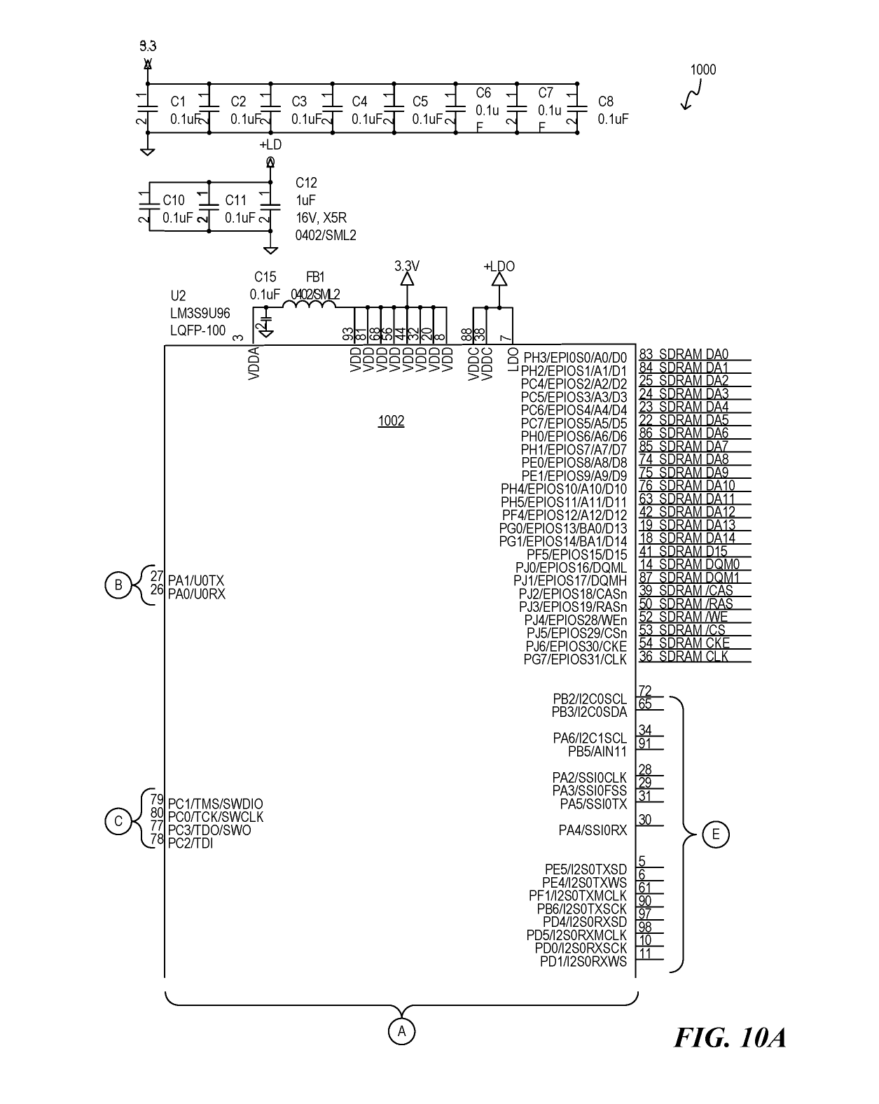

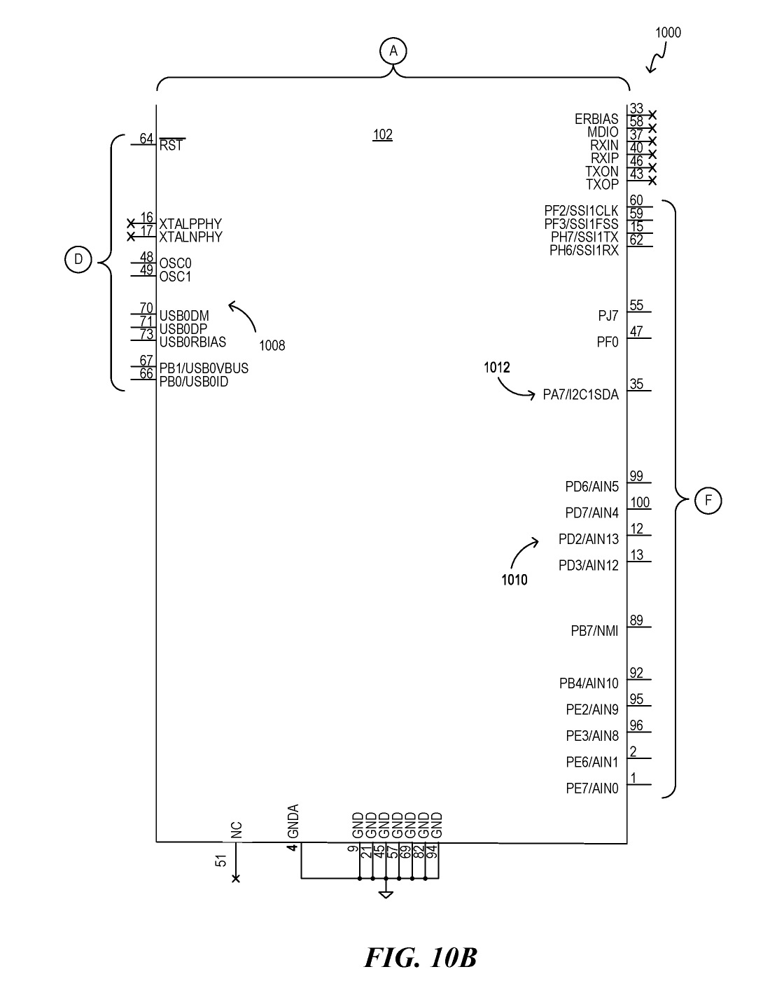

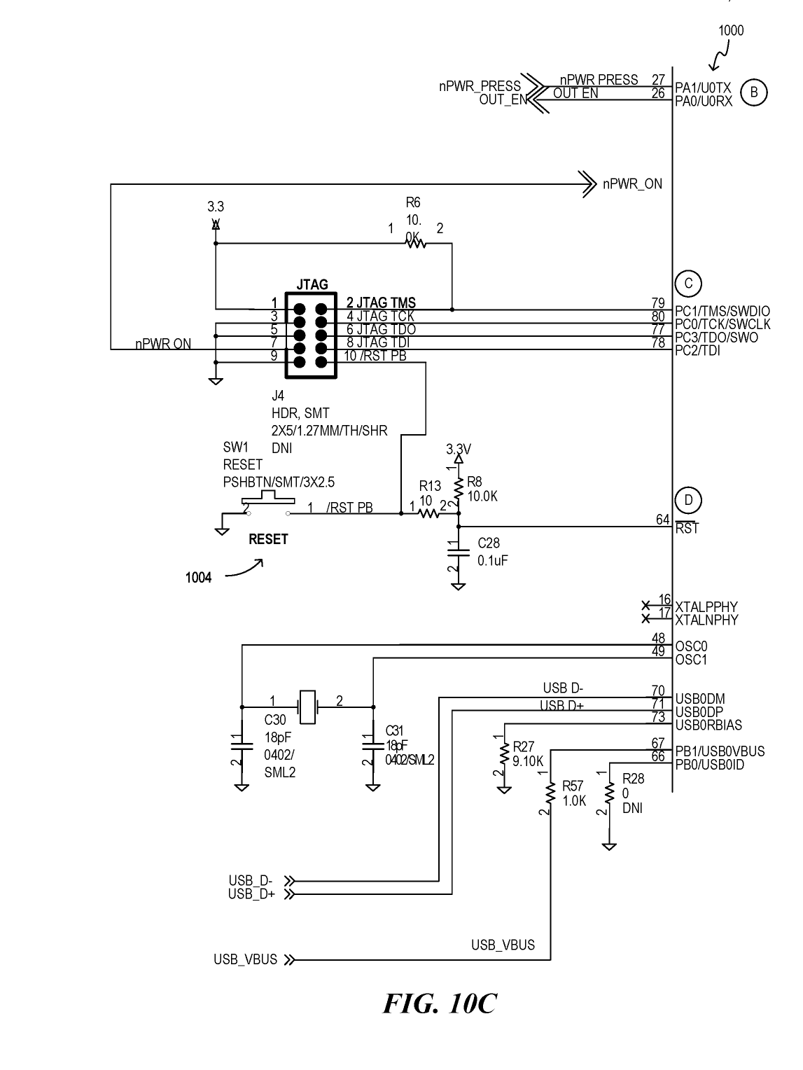

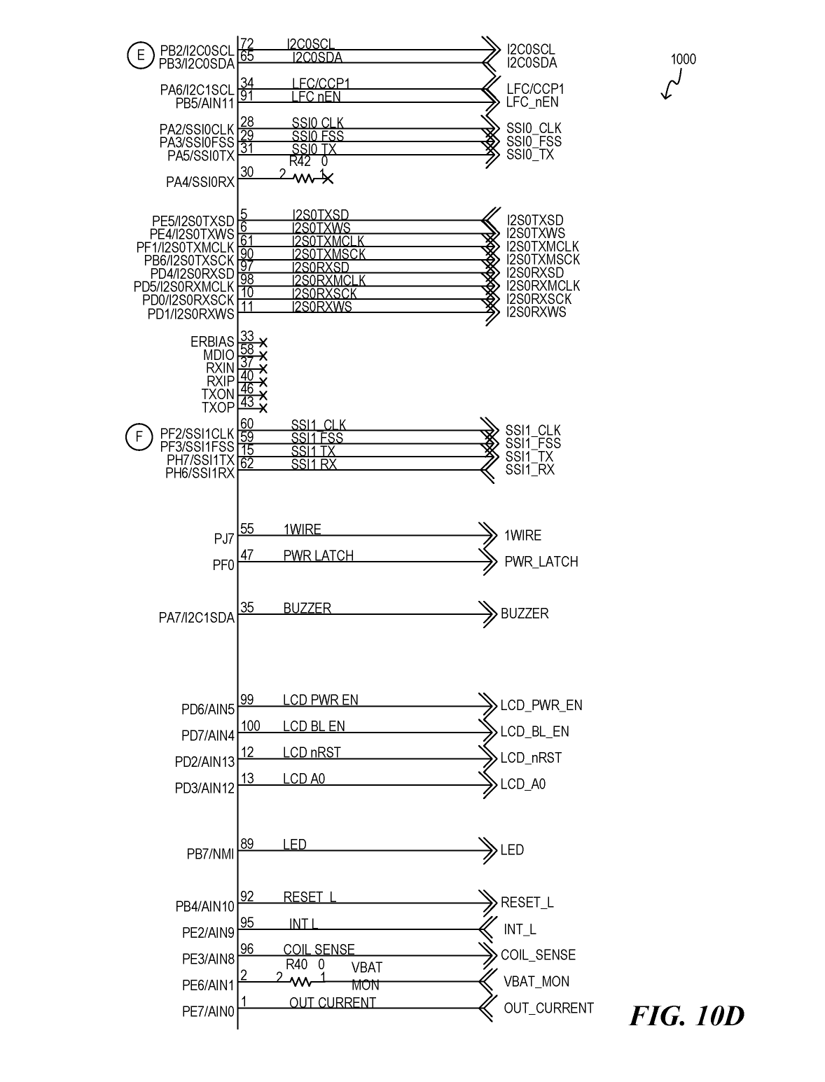

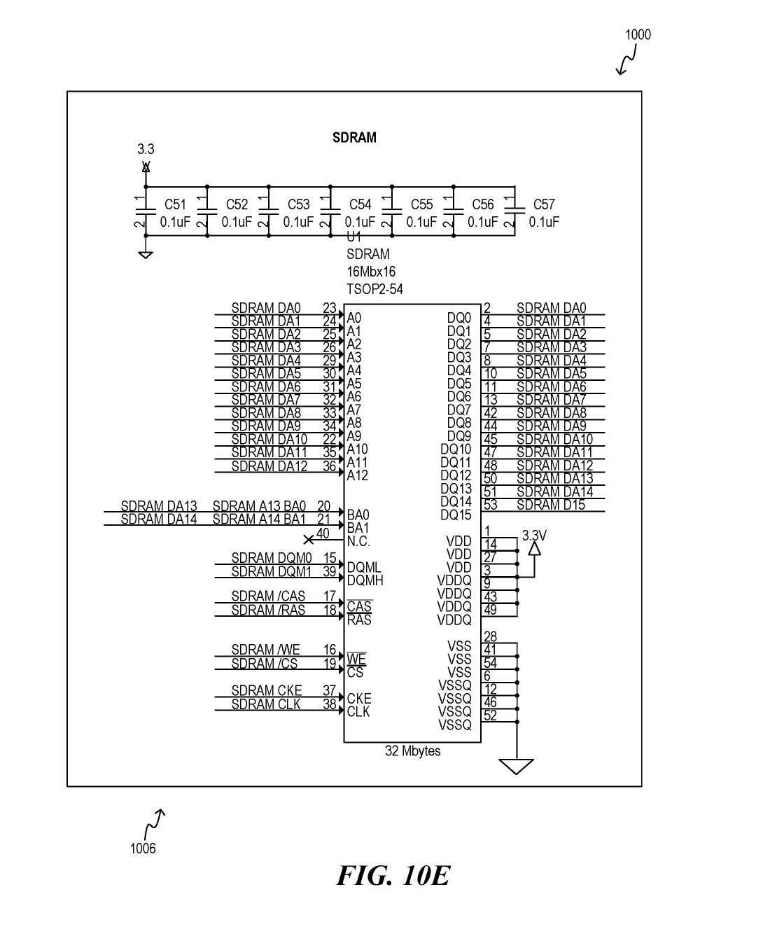

FIGS. 10A-15B illustrate schematics of electronics that the controller 104 may include to perform the various functions described above. The various electronics may be integrated into one or more programmable controllers or may include discrete electronic components electrically and communicatively coupled to each other.

FIGS. 10A-10E illustrate microcontroller circuitry 1000 for operating the controller 104. The circuitry 1000 includes a microprocessor 1002, a reset circuit 1004, and a volatile memory 1006. The microcontroller may be a standard microprocessor, microcontroller or other similar processor, or alternatively be a tamper-resistant processor to improve security. The microprocessor 1002 may include a number of analog and/or digital communication pins to support communications with electronics that are both external and internal to the housing 900. The microprocessor 1002 may include USB pins 1008 to support communication via the USB protocol, display pins 1010 to communicate with a visual interface, and audio pins 1012 to provide an audio interface, in addition to other data communication pins.

Microcontroller 1002 can be configured to use the USB pins 1008 to securely receive cognate files from one or more external devices. Encryption of the cognate file may increase security of the contents of the cognate file. Encryption systems regularly suffer from what is known as the key-distribution-problem. The standard assumption in the cryptographic community is that an attacker will know (or can readily discover) the algorithm for encryption and decryption. The key is all that is needed to decrypt the encrypted file and expose its intellectual property. The legitimate user of the information must have the key. Distribution of the key in a secure way attenuates the key-distribution-problem.

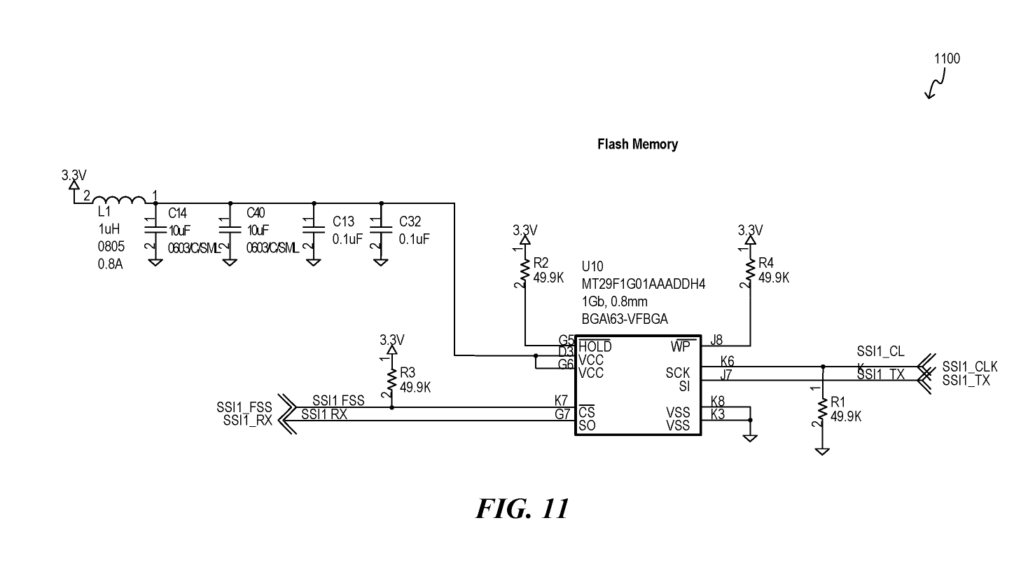

In some embodiments, the microcontroller 1002 is configured to use the Advanced Encryption Standard (AES). AES is a specification for the encryption of electronic data established by the U.S. National Institute of Standards and Technology (NIST) and is used for inter-institutional financial transactions. It is a symmetrical encryption standard (the same key is used for encryption and decryption) and can be secure while the key distribution security is maintained. In some implementations, the microcontroller 1002 uses a 128-bit AES key that is unique to each controller and is stored in non-volatile memory 1100 (illustrated in FIG. 11). The encryption key can be random to reduce the likelihood of forgery, hacking, or reverse engineering. The encryption key can be loaded into non-volatile memory 1100 during the manufacturing process or before the controller is delivered to users. Using AES encryption, the ulRFE® signal can be encrypted and uploaded to one or more servers to facilitate selective delivery to various controllers 104. For example, an agricultural professional may obtain authorization to download cognates to controllers for his/her application. When the agricultural professional contacts and logs in to a server to obtain a cognate, the professional may first need to provide some information, e.g., may need to identify the target device (the controller), for the server (e.g., by a globally unique ID (GUID) stored in the controller) so that the server can look up the target device in a database and provide a cognate file that is encrypted with a key that is compatible with the controller. The encrypted cognate file can then be loaded into the non-volatile memory 1100 via the microcontroller 1002, using USB or another communications protocol. Alternatively, or additionally, the encrypted cognate file may be stored directly to the non-volatile memory 1100 during the manufacturing process to reduce the likelihood of interception of the cognate file, and before the front and back portions of the housing are sealed together.

The microcontroller 1002 can also be configured to log use of the system 100. The log can be stored in a non-volatile memory 1100 and downloaded when a user delivers a controller 104 back to the device distributor, e.g., after the prescribed time allotment for the controller 104 has depleted. The log can be stored in a variety of data formats or files, such as, separated values, as a text file, or as a spreadsheet to enable the display of activity reports for the controller 104. In some implementations, the microcontroller 1002 is configured to log information related to errors associated with coil connections, electrical characteristics of the coil over time, dates and times of use of the system, battery charge durations and discharge traditions, and inductance measurements or other indications of a coil being placed in contact with a human, a mammal, and/or an animal. The microcontroller 1002 can provide log data or the log file to a system monitor using a USB port or other mode of communication to allow the monitor to evaluate the quality and/or function of the system and the quantity and/or use of the system. Notably, the microcontroller 1002 can be configured to log any disruptions in cognate delivery and can log any errors, status messages, or other information provided to the user through user interface of the controller 104 (e.g., using the LCD screen).

The microcontroller 1002 can be configured to use the volatile memory 1006 to protect the content of the cognate file. In some implementations, the cognate file is encrypted when the microcontroller 1002 transfers the file from an external source into non-volatile memory 1100. The microcontroller 1002 can then be configured to only store decrypted versions of the content of the cognate file in volatile memory 1006. By limiting the storage of decrypted content to volatile memory 1006, the microcontroller 1002 and thus the controller 104 can ensure that decrypted content is lost when power is removed from the microcontroller circuitry 1000.

The microcontroller 1002 can be configured to execute additional security measures to reduce the likelihood that an unauthorized user will obtain the contents of the cognate file. For example, the microcontroller 1002 can be configured to only decrypt the cognate file after verifying that an authorized or legitimate coil and cable assembly 102 has been connected to the controller 104. As described above, the coil and cable assembly 102 may include an integrated circuit that may store one or more encrypted or not encrypted identifiers for the coil and cable assembly 102. In some implementations, the microcontroller 1002 is configured to verify that an authorized or prescribed coil and cable assembly 102 is connected to the controller 104. The microcontroller 1002 may verify the authenticity of a coil and cable assembly 102 by comparing the identifier from the integrated circuit of the coil and cable assembly 102 with one or more entries stored in a lookup table in either volatile memory 1006 or non-volatile memory 1100. In other implementations, the microcontroller 1002 may be configured to acquire a serial number of the integrated circuit and measure electrical characteristics of the coil and cable assembly 102 and perform a cryptographic function, such as a hash function, on a combination of the serial number and the electrical characteristics. Doing so may deter or prevent an unauthorized user from copying the contents of the integrated circuit of the coil and cable assembly 102 into a duplicate integrated circuit associated with an unauthorized copy of a coil and cable assembly.

The microcontroller 1002 can be configured to delete the cognate file from volatile memory 1006 and from non-volatile memory 1100 in response to fulfillment of one or more predetermined conditions. For example, the microcontroller 1002 can be configured to delete the cognate file from memory after the controller has delivered the prescribed drug-simulating signals for a specific period of time, e.g., 14 days. In other embodiments, the microcontroller 1002 can be configured to delete the cognate file from memory after the controller detects a coupling of the controller 104 with an unauthorized coil and cable assembly. The microcontroller 1002 can be configured to delete the cognate file after only one coupling with an unauthorized coil and cable assembly, or can be configured to delete the cognate file after a predetermined number of couplings with an unauthorized coil and cable assembly. In some implementations, the microcontroller can be configured to monitor an internal timer and delete the cognate file, for example, one month, two months, or longer after the cognate file has been installed on the controller 104.

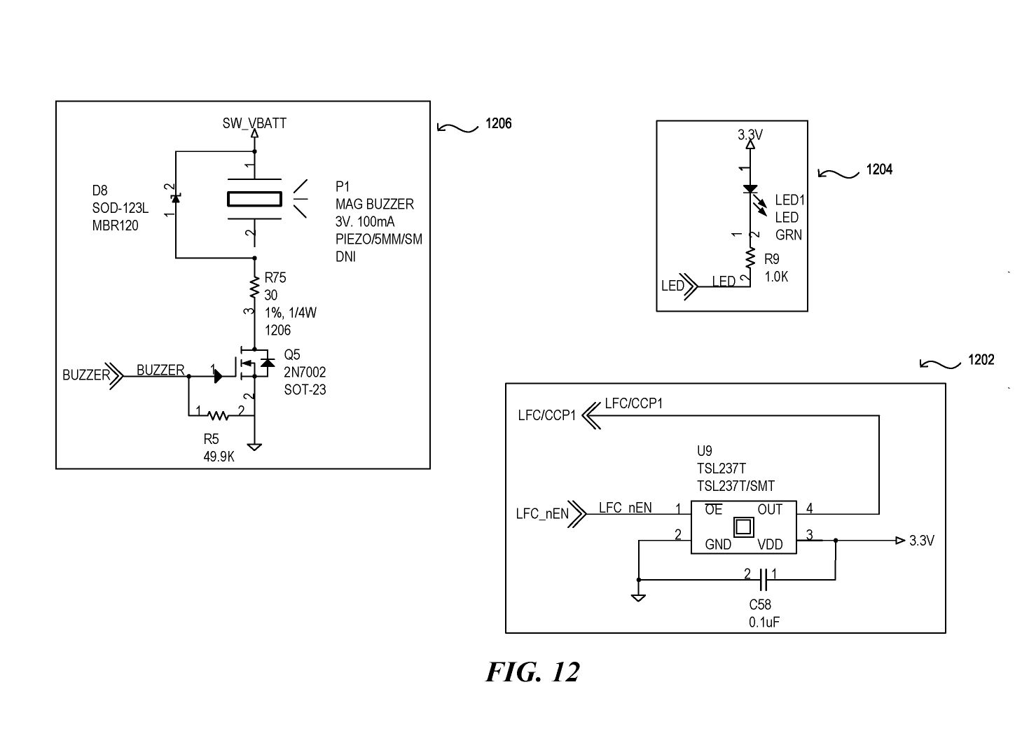

The microcontroller 1002 can be configured to delete the cognate file from volatile memory 1006 and from non-volatile memory 1100 in response to input from one or more sensors. FIG. 12 illustrates a sensor 1202 that may provide a signal to the microcontroller 1002 in response to a physical disruption of the housing 900 of the controller 104. For example, the sensor 1202 can be a light sensor that detects visible and non-visible wavelengths within the electromagnetic spectrum. For example, the sensor 1202 can be configured to detect infrared, visible light, and/or ultraviolet light. Because the detection of light within the housing 900 can be an indication of intrusion into the housing 900, the microcontroller 1002 can be configured to delete and/or corrupt the cognate file upon receipt of a signal from the sensor 1202. In some implementations, the sensor 1202 is a light sensor. In other implementations, the sensor 1202 can be a pressure sensor, a capacitive sensor, a moisture sensor, a temperature sensor, or the like.

In response to detection of unauthorized use of the controller 104, or to increase the user-friendliness of the system 100, the microcontroller 1002 can use various indicators or interfaces to provide information to a user. As examples, FIG. 12 illustrates an LED 1204 and an audible buzzer 1206. The microcontroller 1002 can illuminate the LED 1204 and/or actuate the audible buzzer 1206 in response to user error, unauthorized tampering, or to provide friendly reminders of deviation from scheduled use of the system 100. Although one LED is illustrated in the LED 1204, multiple LEDs having various colors can also be used. Additionally, although the audible buzzer 1206 is described as a buzzer, in other implementations, the audible buzzer 1206 can be a vibrating motor, or a speaker that delivers audible commands to facilitate use of the system 100 by sight impaired users.

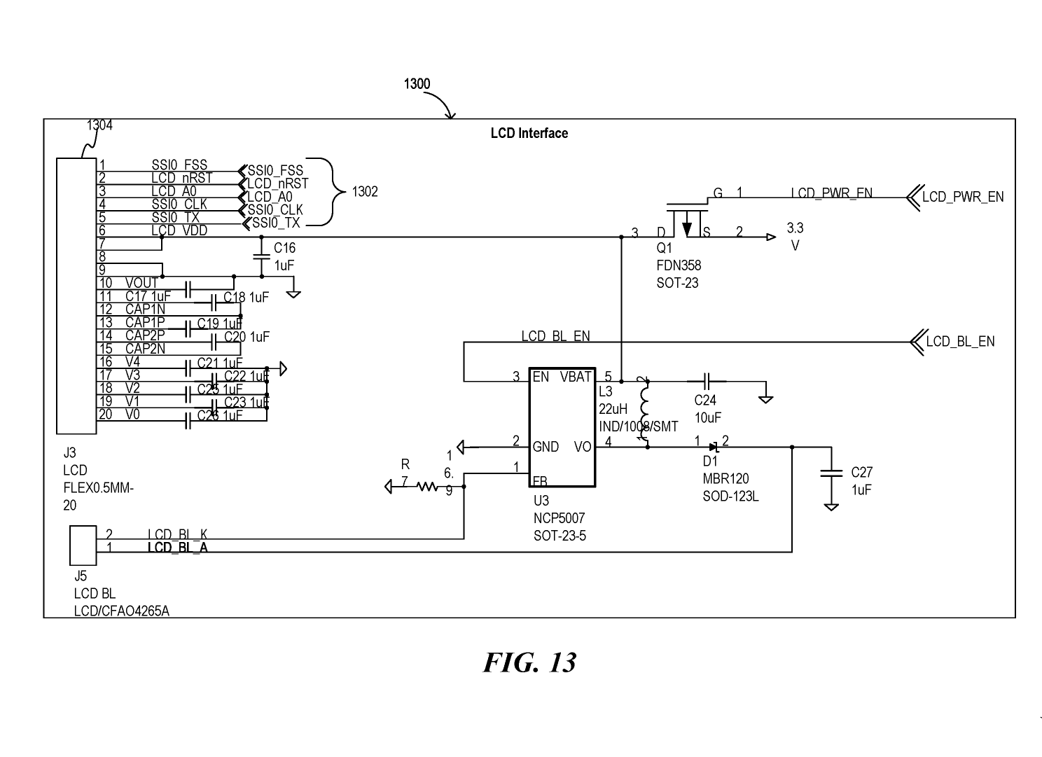

FIG. 13 illustrates an LCD interface 1300 that the microcontroller 1002 can manipulate to interact with a user. The LCD interface 1300 can receive various commands from the microcontroller 1002 at input pins 1302. In response to inputs received from the microcontroller 1002, an LCD screen 1304 can be configured to display various messages to a user. In some implementations, the LCD screen 1304 displays messages regarding battery status, duration of prescription use or exposure, information regarding the type of prescription being administered, error messages, identification of the coil and cable assembly 102, or the like. For example, the LCD screen 1304 can provide a percentage or a time duration of remaining battery power. The LCD screen 1304 can also provide a text-based message that notifies the user that the battery charge is low or that the battery is nearly discharged. The LCD screen 1304 can also be reconfigured to provide a name of a prescription or exposure period (e.g., corresponding name of the physical drug, chemical or other agent) and/or a human, a mammal, and/or an animal part for which the prescription or exposure is to be used. The LCD screen 1304 can also provide notification of elapsed-time or remaining-time for administration of a prescription or exposure. If no additional prescription or exposure time is authorized, the LCD screen 1304 can notify the user to contact the applicable prescriber or provider.

The LCD screen 1304 can be configured to continuously or periodically provide indications regarding the status of the connection between a coil and the controller. In some implementations, the LCD screen 1304 can be configured to display statuses or instructions such as, “coil connected,” “coil not connected,” “coil identified,” “unrecognized coil,” “reconnect coil,” or the like. In some implementations, the LCD screen 1304 can provide a graphical representation of a coil and flash the coil when the coil is connected properly or improperly. Alternatively, or additionally, the controller can monitor an impedance from the coil to detect a change, a possible removal, or loss of the coil from the area to be treated, and provide a corresponding error message. The LCD interface 1300, in other implementations, can be a touch screen that delivers information to the user in addition to receiving instructions or commands from the user. In some implementations, the microcontroller 1002 can be configured to receive input from hardware buttons and switches to, for example, power on or power off the controller 104. The switch on the device permits an on-off nature of treatment so that treatment may selectively be switched on and off if needed.

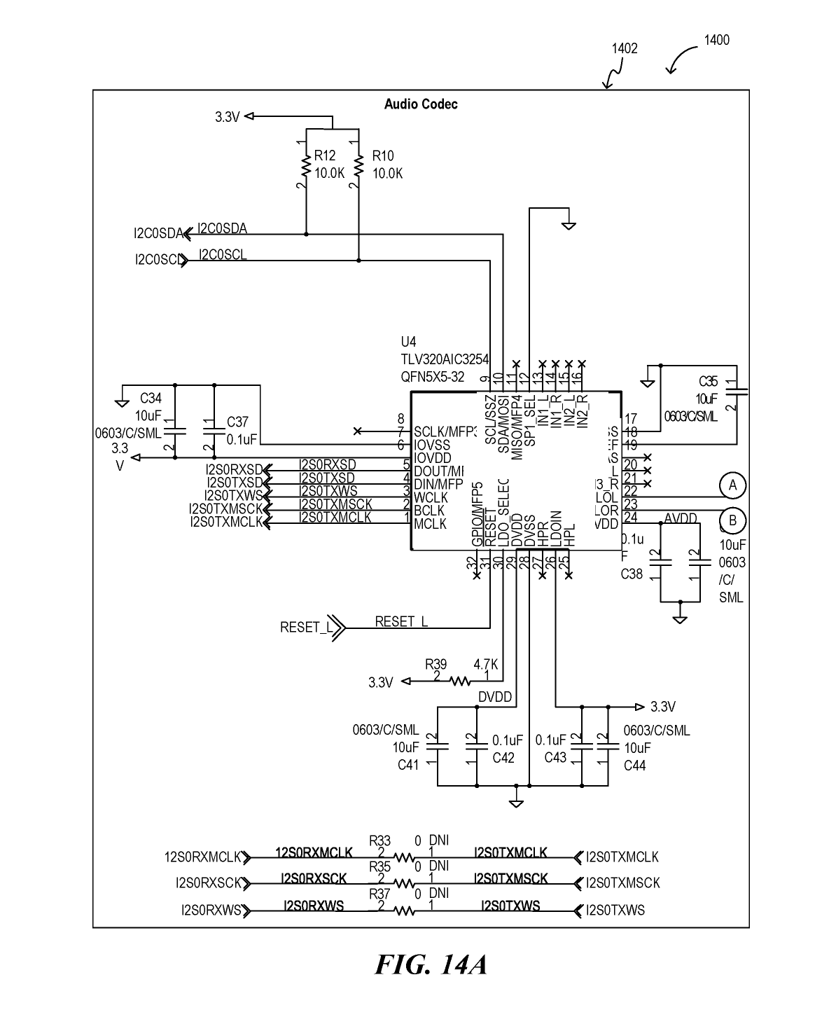

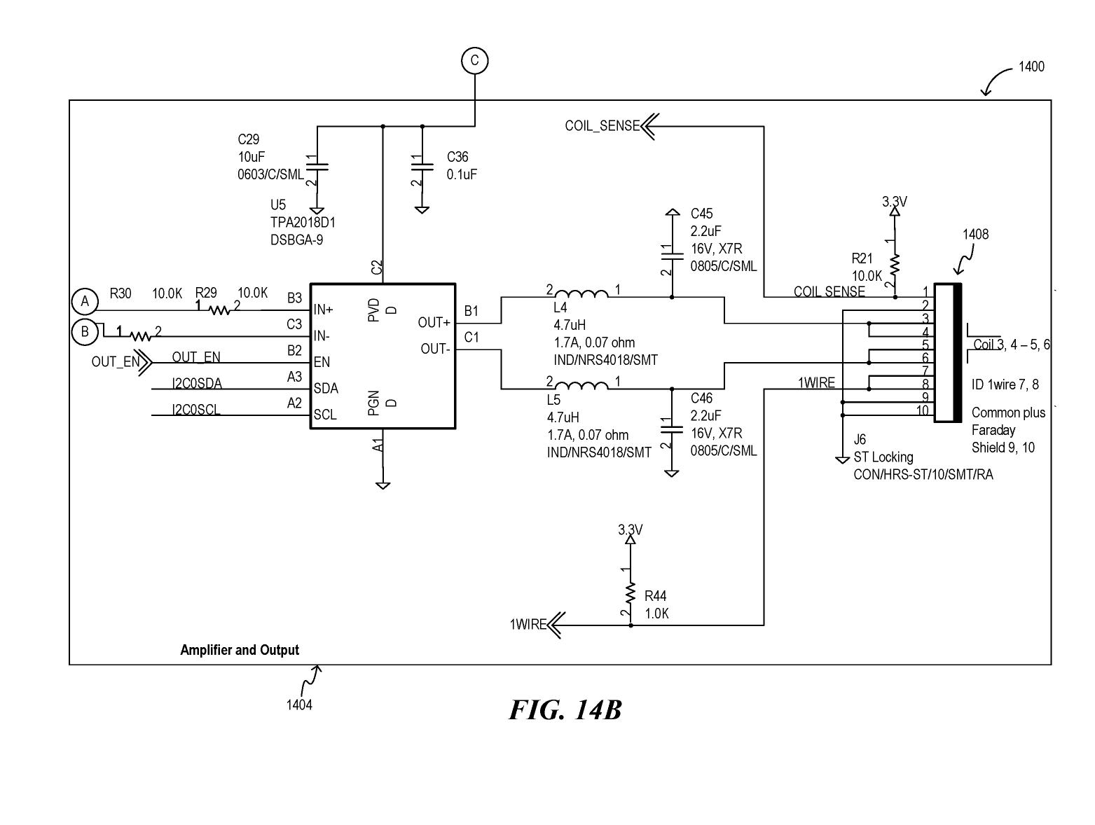

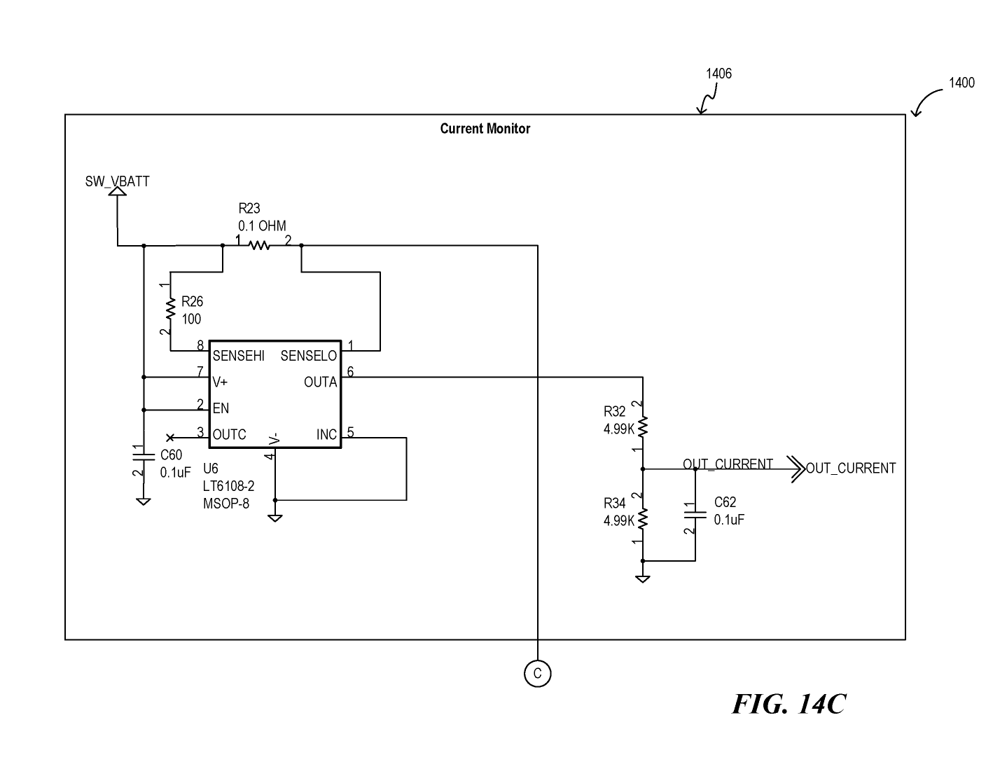

FIGS. 14A-14C illustrate signal generation circuitry 1400 that may be used to drive the coil and cable assembly 102 with drug or chemical-simulating signals. The circuitry 1400 may include an audio coder-decoder 1402, and output amplifier 1404, and a current monitor 1406. The audio coder-decoder 1402 may be used to convert digital inputs received from volatile memory 1006, non-volatile memory 1100, or from microcontroller 1002 into analog output signals useful for driving the coil and cable assembly 102. The audio coder-decoder 1402 may be configured to output the analog output signals to the output amplifier 1404. In some implementations, the output amplifier 1404 is programmable so that the intensity or amplitude of the signals transmitted to the coil may be varied according to the treatment prescribed for the human, mammal, and/or animal.

Because the controller 104 can be connected with coils having different sizes, shapes, and numbers of windings, the output amplifier 1404 can be configured to adjust the intensity level of the ulRFE® cognates delivered to the coil so that each coil delivers a drug or chemical-simulating ulRFE® cognate that is uniform between different coils, or different between coils, for a particular prescription or exposure period. The coil dimensions and electrical characteristics influence the depth and breadth of the magnetic field, so programmatically adjusting the output intensity of the output amplifier 1404 to deliver uniform drug or chemical-simulating ulRFE® cognates can advantageously enable the selection of a coil that is appropriate for a particular treatment area, to avoid inadvertently altering the prescription or exposure period. As described above, the controller 104 can determine the dimensions and electrical characteristics of a coil by reading such information from the integrated circuit 610 (shown in FIGS. 6 and 7). The cognate generation circuitry 1400 can be configured to use the dimensional and electrical characteristic information acquired from the coil to programmatically adjust the level of intensity of ulRFE® output by the output amplifier 1404.

The output amplifier 1404 may include a low pass filter that significantly reduces or eliminates ulRFE® output having a frequency higher than, for example, 50 kHz. In other implementations, the low pass filter can be configured to significantly reduce or eliminate ulRFE® output having a frequency higher than 22 kHz. The cognate generation circuitry 1400 may use the current monitor 1406 to determine electrical characteristics of the coil and cable assembly 102 and/or to verify that ulRFE® output levels remain within specified thresholds. The ulRFE® cognate generation circuitry 1400 may also include a connector 1408 that mates with the connector 206 of the coil and cable assembly 102. The connector 1408 can provide the electrical interface between the microcontroller 1002 and the coil and cable assembly 102.

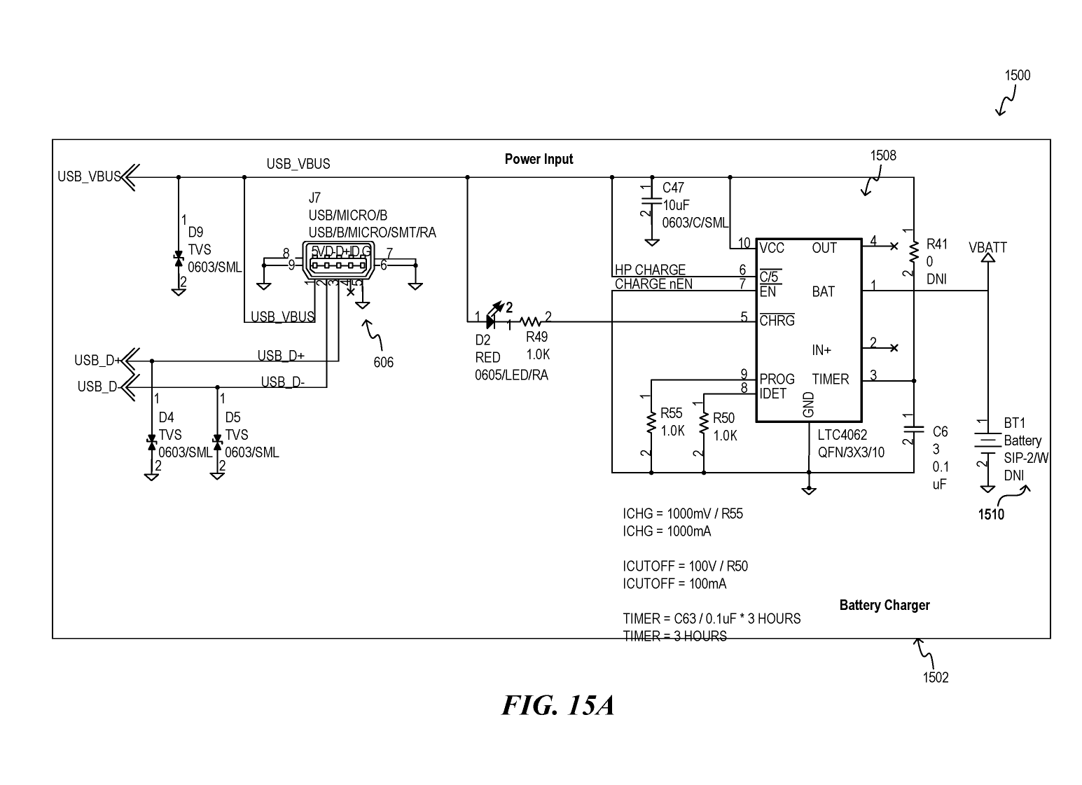

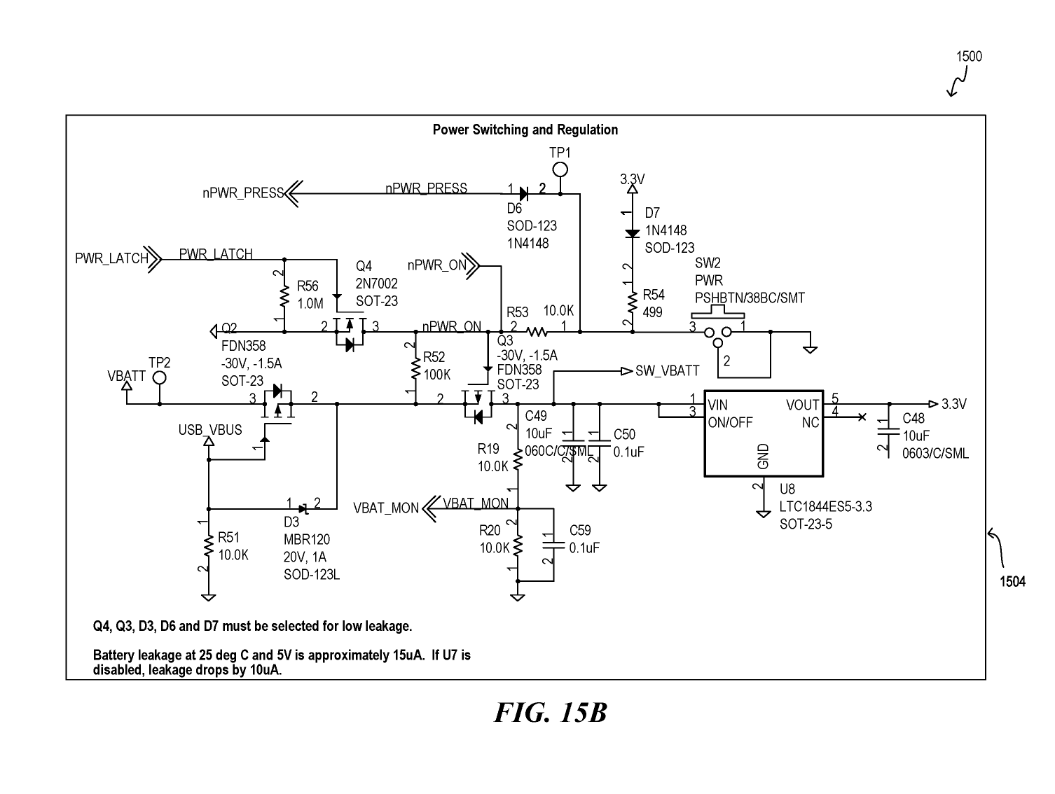

FIGS. 15A-15B illustrate power control circuitry 1500 for receiving and regulating power to the controller 104. The power control circuitry 1500 includes power input circuitry 1502 and power regulation circuitry 1504. The power input circuitry 1502 can include a connector 1506, e.g., a micro-USB connector, to receive power from an external source for recharging a battery 1510. The power input circuitry 1502 can also include a charging circuit 1508 that monitors a voltage level of the battery 1510 and electrically decouples the battery from the connector 1506 when the battery 1510 is sufficiently charged. The power regulation circuitry 1504 can be used to convert a voltage level of the battery 1510 to a lower voltage for use by the various circuits of the controller 102. For example, when fully charged, the battery 1510 may have a voltage of about 4.2 to 5 volts, whereas the microcontroller may have an upper voltage threshold of 3.5 volts. The power regulation circuitry 1504 can be configured to convert the higher voltage of the battery, e.g., 4.2 volts, to a lower voltage, e.g., 3.3 volts, that is usable by the electronic devices of the controller 102.

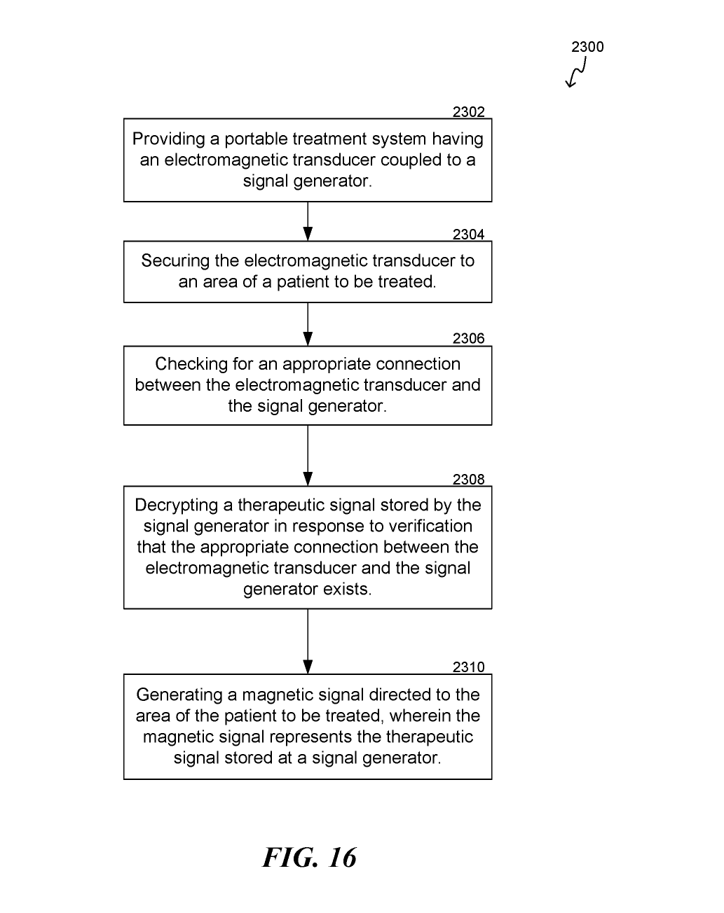

FIG. 16 illustrates a method 1600 of operating a portable system that may be used to provide magnetic field treatment that is non-invasive, non-thermal, non-ionizing and mobile.

At block 1602 an electromagnetic transducer is coupled to a ulRFE® cognate generator. The electromagnetic transducer can be a coil having various shapes and sizes according to the size of the object or condition to be treated.

At block 1604 the electromagnetic transducer is secured to an area of the animal to be treated. The transducer may be secured using elastic bandages, gauze, tape, or the like.

At block 1606, the ulRFE® cognate generator checks for an appropriate connection to the electromagnetic transducer. The ulRFE® cognate generator can be configured to verify an identification or electrical characteristics of the electromagnetic transducer, such as a resistance or impedance of the transducer to ensure that an appropriate transducer is coupled to the generator. In some implementations, the ulRFE® cognate generator can be configured to periodically monitor the electrical characteristics of the electromagnetic transducer to ensure that an appropriate connection is maintained. For example, if the ulRFE® cognate generator detects an increase in resistance or decrease in inductance, the ulRFE® cognate generator may be configured to cease delivery of ulRFE® to the electromagnetic transducer. The ulRFE® cognate generator may cease delivery of ulRFE® when unexpected electrical characteristics are detected to protect the health and/or safety of the subject or to protect the subject being treated, and to prevent unauthorized attempts to acquire generated ulRFE® cognates. As discussed above, the ulRFE® cognate generator may be configured to log the periodic checks of the electrical characteristics of the electromagnetic transducer and can provide the log data for review. Other security checks may be performed as described herein.

At block 1608 the ulRFE® cognate generator decrypts a ulRFE® cognate stored by the ulRFE® cognate generator in response to verification that an appropriate connection between the electromagnetic transducer and the ulRFE® cognate generator exists. Where the term “ulRFE® cognate” is used herein, the term generally applies to any stored cognate that the disclosed system uses to induce a chemical, biological or other change in a biological system.

At block 1610 the electromagnetic transducer generates a ulRFE® cognate directed to the human, mammal, and/or animal or specific anatomical region of the human, mammal, and/or animal to be treated. The cognate used to generate the specific electromagnetic field is stored in the ulRFE® cognate generator. According to various implementations, the cognate's magnetic field has a frequency in the range of 0 Hz to 22 kHz.

In some instances, the ulRFE® cognate can be delivered to a subject (e.g., human, mammal, and/or animal) in addition to administering a drug, chemical or other agent to the subject. For example, the drug, chemical or other agent can be administered and/or applied to human, mammal, and/or animal, or area of the human, mammal, and/or animal to be treated with the ulRFE® cognate before or after the ulRFE® cognate is delivered to the subject. In some instances, the ulRFE® cognate is derived from a sample of the same drug, chemical or other agent administered to the subject. In other instances, the ulRFE® cognate derived from a sample of a different drug, chemical or other agent than that administered to the subject. Moreover, the drug, chemical or other agent and/or the ulRFE® cognate can be delivered to the subject more than once and in any sequence, for example, drug, chemical or other agent+ulRFE® cognate+drug, chemical or other agent, or ulRFE® cognate+drug, chemical or other agent+ulRFE® cognate, etc. In further instances, the sequences can include more than one ulRFE® cognate and more than one drug, chemical or other agent.

In some implementations, the cognate of a sample of a drug, chemical or other agent may be acquired by placing the sample in an electromagnetic shielding structure and by placing the sample proximal to, at least one, superconducting quantum interference device (SQUID) (or magnetometer). The drug, chemical or other agent sample is placed in a container having both magnetic and electromagnetic shielding, where the sample drug, chemical or other agent acts as a signal source to record the ulRFE® molecular cognate. In some embodiments, noise is injected into the drug, chemical or other sample at a noise amplitude sufficient to generate stochastic resonance, where the noise has a substantially uniform amplitude over multiple frequencies. The stochastic resonance induced by noise injection may allow an otherwise undetectable signal to be recorded. Using the superconducting quantum interference device (SQUID) (or the magnetometer), the electrostatic surface potential of the drug, chemical or other agent sample is detected and recorded as an electromagnetic time-domain signal composed of sample-source radiation superimposed on the injected noise (if any). The recording of an electromagnetic time-domain signal from a sample may be repeated at multiple noise levels to enable the detection of a sample-specific signal.

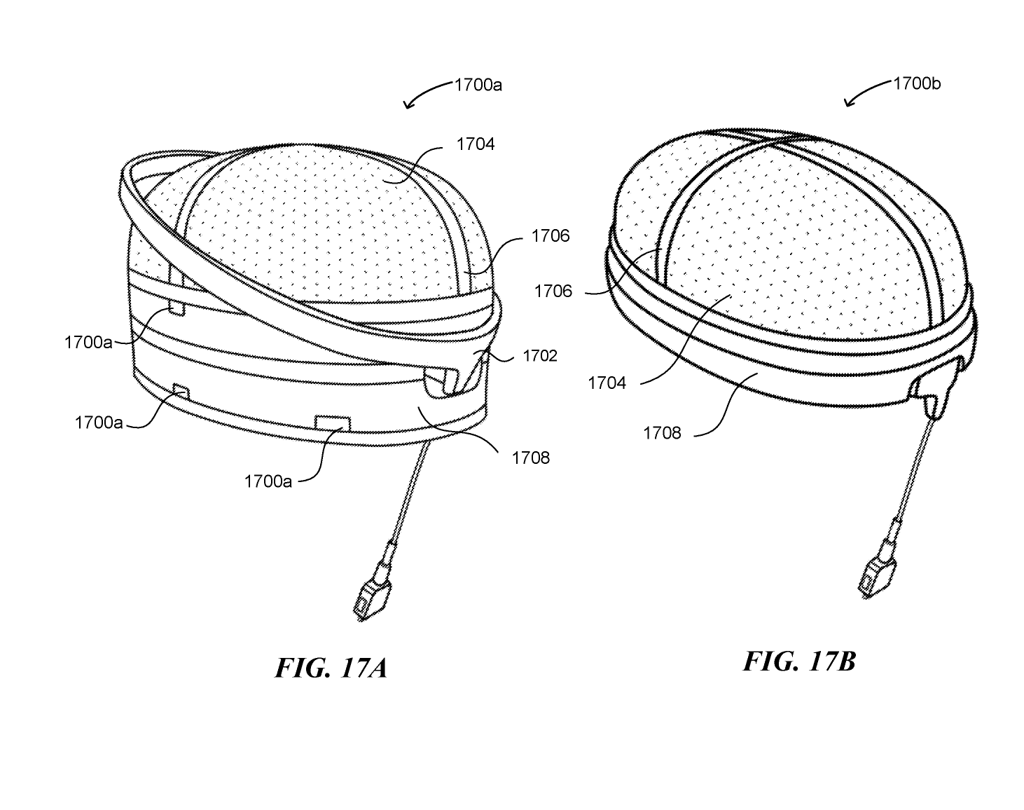

FIGS. 17A and 17B illustrate example embodiments of headgear 1700 (inclusive of 1700a and 1700b) that may be used to position or secure a coil 1702 around the cranium of an animal. The headgear can include a breathable mesh 1704, elastic straps 1706, and a band 1708. The breathable mesh 1704, elastic straps 1706, and the band 1708 can provide a comfortable apparatus for carrying, securing, or otherwise positioning the coil 1702 around the cranium of an animal. The headgear 1700 may also include fasteners 1710 (inclusive of 1710a, 1710b, 1710c) for securing the band 1708 over the coil 1702. The fasteners 1710 may be influenced with Velcro, snaps, or other types of securing devices. In FIG. 17A, the headgear 1700a illustrates the coil 1702 in an exposed or unsecured position. In FIG. 17B, the headgear 1700b illustrates the coil 1702 in a secured position.

### EXAMPLES

The following examples are illustrative of several embodiments of the present technology.

### Example 1: Treatment of rGBM Using ulRFE® Signal Alone or in Combination with a Chemotherapy or Avastin®

This example demonstrates that the Nativis Voyager® system is feasible and safe for the treatment of rGBM. The therapy was delivered non-invasively, and no serious adverse events attributed to the therapy were reported.

As described above, systems configured in accordance with the present technology can record the dynamic magnetic field of one or more molecules in aqueous solution. One or more of these systems can transmit the recorded magnetic field information (e.g., cognate, signal, etc.) to an in vitro or in vivo system, such as cells or a living subject. The Nativis Voyager® ulRFE® system, a non-invasive device, was studied in a first-in-human feasibility study to assess safety and feasibility of the treatment for rGBM using a cognate derived from paclitaxel.

In the present example, the Voyager system was used to administer ulRFE® to a group of subjects with rGBM. Some subjects were being simultaneously treated with chemotherapy or Avastin® at the discretion of the doctor.

In a multi-center trial, patients with GBM who had exhibited recurrence after receiving standard-of-care chemotherapy and/or radiotherapy were considered for the study. Safety was assessed by incidence of any adverse events associated with the investigational therapy.

Tumor progression at eight weeks (two cycles) was assessed by radiological response by a local clinical site. Patients were followed at least every eight weeks during treatment and every four months thereafter. Fourteen patients were enrolled and treated in the United States. Eleven subjects were followed per protocol in the first stage of a two-stage study. Three subjects withdrew consent prior to the first radiological assessment (day 28) for reasons not associated with the study or investigational therapy and were not included in the analysis. The local clinical site reported a partial response in the first two months of treatment in two of the eleven subjects. One of these two subjects received ulRFE® and the other received ulRFE® in combination with chemotherapy lomustine (CCNU). Both subjects were Avastin®-naïve. Two subjects were reported to be progression free after 6 cycles (24 weeks) of treatment, one subject received ulRFE® and the other subject received ulRFE® in combination with lomustine (CCNU). No serious adverse events associated with the therapy were reported.

### Example 2. Treatment of rGBM Using an ulRFE® Signal

This is another example demonstrating that the ulRFE® signal delivered by the Nativis Voyager® system is feasible and safe for the treatment of rGBM. The ulRFE® signal was delivered non-invasively, and no serious adverse events attributed to the ulRFE® signal were reported.

As described above, systems configured in accordance with the present technology can record the dynamic magnetic field of one or more molecules in aqueous solution. One or more of these systems can transmit the recorded magnetic field information (e.g., ulRFE®, cognate, signal, etc.) to an in vitro or in vivo system, such as cells or a living subject safely and non-invasively. The Nativis Voyager® ulRFE® system, a non-invasive device, was studied in a first-in-human feasibility study to assess safety and feasibility of the Nativis Voyager® ulRFE® system for delivery of a ulRFE® signal for treatment of rGBM. The ulRFE® signal is either the anti-mitotic therapy (e.g., AIA ulRFE® signal derived from taxane) or the anti-CTLA-4/anti-PD-1 therapy (e.g., A2HU ulRFE® signal derived from CTLA-4 siRNA and PD-1 siRNA) and is delivered to the in vitro or in vivo system, such as the living subject's cranium (e.g., brain).

In the present example, the Voyager system was used to administer an AIA ulRFE® signal to a group of subjects with rGBM. Patients with rGBM who had exhibited recurrence after receiving standard-of-care chemotherapy and radiotherapy were considered for the study. Nineteen patients were initially enrolled. After evaluation, sixteen patients were treated with Voyager monotherapy. Safety was assessed by incidence of any adverse events associated with the investigational therapy.

Tumor progression at eight weeks following two cycles of ulRFE® therapy was assessed by radiological response at the subject's local site. Patients were followed at least every eight weeks during treatment and every four months thereafter. Five patients were enrolled and treated per protocol using the Voyager system utilizing a single signal and eleven patients were treated per protocol using the Voyager system utilizing two signals. The clinical site reported two patients to be progression free after 6 cycles over the course of 24 weeks of ulRFE® anti-mitotic treatment using the AIA ulRFE® signal and one patient to be progression free after 6 cycles of ulRFE® anti-CTLA-4/anti-PD-1 treatment using the A2HU ulRFE® signal. No serious adverse events associated with the therapy were reported.

### Example 3. Nativis Voyager® System in Patients with rGBM Using AIA ulRFE® and A2HU ulRFE®

This is yet another example that demonstrates that the Nativis Voyager® system is feasible and safe for the treatment of rGBM. The therapy was delivered non-invasively, and no serious adverse events attributed to the therapy were reported.

The Nativis Voyager® system used in this example is described above and the objective of this study was to assess whether the Voyager ulRFE® therapy was a safe and feasible treatment for rGBM. Feasibility studies of the Nativis Voyager® system in patients with rGBM were conducted in the United States and in Australia as described below.

Materials and Methods—Patient Selection and Study Design:

Subjects were eligible to participate in the study if they had a histologically-confirmed diagnosis of GBM, failed or were intolerant to radiotherapy, failed or were intolerant to temozolomide therapy, had progressive disease with at least one measurable lesion on MRI or CT, were at least 18 years of age, had a KPS score≥60, had adequate organ and marrow function, and provided signed, informed consent

The Nativis Voyager® system is a non-sterile, non-invasive, non-thermal, non-ionizing, portable medical device that uses localized ulRFE® in the range 0 to 22 kHz for the treatment of malignant solid tumors. The ulRFE® was delivered to the patient by an electromagnetic coil worn externally on the head. The system consisted of 3 components: a battery-operated controller, an electromagnetic coil, and a battery charger. The coil was worn on the head much like a crown and was connected to the controller via a cable. No special alignment of the coil was necessary. The device was worn continuously except for brief periods for medical procedures and personal hygiene. Patients were provided 2 controller units to make a fully-charged unit always available. Treatment was administered continuously until unequivocal disease progression, occurrence of a device-related clinically significant adverse event, unacceptable adverse reactions, or removal from the study. At the discretion of the investigator, patients could remain on treatment post-progression. Patient visits occurred at least every 8 weeks during the first 6 months and every 4 months thereafter. Routine hematology and chemistry assessments, a physical exam including vital signs and neurological exam, and MRI were performed at baseline and at each visit.

The Voyager controller can produce a variety of cognates, although cognates were factory-set and not user-adjustable. Patients were expected to wear the device continuously while on study, except for brief periods of less than one hour as necessary for personal hygiene or medical procedures.

As stated above, the objective of this study was to assess whether the Voyager ulRFE® therapy was a safe and feasible treatment for rGBM. The Voyager system was contemplated to be at least comparable to other therapies with fewer side effects and improved quality of life.

In this multi-site, prospective, open-label trial, conducted in the United States, patients with rGBM, following standard-of-care chemotherapy and radiotherapy, were considered for the study. Patients were treated with Voyager as monotherapy or in combination with best standard of care (BSC) anti-cancer agents. Safety was assessed by incidence of any adverse events associated with the investigational therapy. Tumor progression at each post-treatment visit was assessed via radiological response by local investigators and by 2 independent radiology reviewers. Patients were followed at least every 8 weeks during the first 6 months and every 4 months thereafter.

In this study, patients received treatment with A1A, a ulRFE® cognate derived from paclitaxel. Investigators were given the choice to treat patients with Voyager alone or to treat with Voyager and BSC anti-cancer agents. The treatment groups were not intended for comparison.

Safety and Clinical Utility Measurements:

Safety was assessed by incidence and evaluation of any adverse events associated with the investigational therapy, abnormal laboratory findings, and abnormal neurological findings. Clinical utility was assessed by tumor response, progression-free survival (PFS) at 6 months, median PFS, overall survival (OS) at several intervals, and median OS. The radiological response of the tumor was assessed by MRI studies according to RANO criteria. All patients had their tumor measurements recorded at baseline and at the time of each MRI scan. The dose and type of contrast agent was held constant from scan to scan for each patient. Images were assessed by the investigators as well as an independent radiology review team.

Statistical Analysis:

The Voyager and Voyager combined with BSC arms were evaluated separately. Data from patients who were enrolled and treated for at least one month were included in the analysis of safety and feasibility in the first cohort.

The data analyses were conducted using SAS® Software, version 9.4 or later. Baseline and demographic characteristics of the safety population were summarized. Continuous variables (age, baseline height) were summarized via mean, standard deviation, median, range, and number of non-missing responses. Categorical variables (gender, race, ethnicity, and KPS) were summarized via counts and percentages.

Adverse events (AEs) were graded according to the NCI Common Terminology Criteria for Adverse Event Version 3.0 (CTCAE V3.0) and were also coded using the Medical Dictionary for Regulatory Activities (MedDRA®). Treatment-emergent AEs (TEAEs), defined as any AE that occurred after a subject received the assigned study treatment, were summarized by the number and proportion of subjects reporting at least one occurrence of the AE. Frequencies of each TEAE were summarized by MedDRA® preferred term within system organ class (SOC), by severity grade, and relation to study device. Treatment emergent serious adverse events (TESAEs) were tabulated by MedDRA® preferred term within SOC.

Clinical laboratory tests were performed at pre-study (baseline) and at all visits. For each panel (hematology, biochemistry, coagulation) the study results were summarized in shift tables from baseline using the categories Normal, Abnormal (Not Clinically Significant), and Abnormal (Clinically Significant). All clinically significant abnormal findings were reported as AEs.

Physical exams, including vital signs and neurological exams, were performed at pre-study (baseline) and at all patient visits. Physical exam shift tables were constructed to summarize the changes in each body system from baseline for each assessed cycle.

Tumor response was assessed by the RANO criteria from MRI conducted at each post-treatment visit. A copy of each scan was submitted to 2 independent radiology reviewers, and outcomes were compared. Patients with unknown status for tumor response at the time point were excluded from the analysis.

Survival rates were estimated using progression free survival (“PFS”) rate at 6 months (PFS-6), overall survival (“OS”) at 6 months (OS-6), OS at 12 months (OS-12), and OS at 24 months (OS-24). Survival rates were summarized by counts (n) and rates (percent surviving to time point) by treatment arm. For the median survival endpoints—i.e., OS (in months) and PFS (in weeks)—patients were followed until death. The start of the efficacy period for all analyses in this study was date of treatment initiation, Day 1. OS was assessed using death as the endpoint. PFS was determined using RANO criteria. A waterfall plot of survival for each treatment arm was produced, displaying survival time and best overall tumor response for each patient

Results

Eighteen patients were screened, and 15 were enrolled and received at least one day of treatment with Voyager ulRFE® AIA signal alone or in combination with BSC. Of these 15-treated patients, 11 were treated for at least one month and were the basis of the safety and feasibility analysis (i.e., the first cohort). The patient disposition (safety population) is enumerated below in Table 1 with one patient still on treatment as of the data cutoff of Jul. 10, 2018.

TABLE 1
Patient Disposition (Safety Population).

| | N = 11 | |
|---|---|---|
| Off Treatment Reasons, n (%) | | |
| Completed Treatment Schedule | 8 | (73%) |
| Documented Disease Progression | 9 | (82%) |
| Treatment Related Toxicity | 0 | (0.0%) |
| Non-treatment Related Toxicity | 0 | (0.0%) |
| Patient Requested Early Discontinuation | 0 | (0%) |
| of Trial but Still Followed | | |
| Physician Requested Early Discontinuation | 2 | (18%) |
| of Trial for Reasons Not Related to Toxicity | | |
| Death | 8 | (73%) |
| Non-Compliance | 0 | (0.0%) |
| Other | 0 | (0%) |
| Off Study Reasons, n (%) | | |
| Completed Treatment Schedule | 0 | (0.0%) |
| Documented Disease Progression | 0 | (0.0%) |
| Patient Requested Early Discontinuation | 0 | (0.0%) |
| of Trial but Still Followed | | |
| Lost to Follow Up | 0 | (0.0%) |
| Death | 8 | (73%) |
| Other | 2 | (18%) |

The patient demographics and other baseline characteristics of the patients are enumerated below in Table 2.

TABLE 2
Demographics and Baseline Characteristics (Safety Population).

| | Treatment Arms | | | |
|---|---|---|---|---|
| | Voyager alone | | Voyager + BSC | |
| Characteristic | (N = 4) | | (N = 7) | |
| Age (years) | | | | |
| Median (Min, Max) | 56.5 | (33, 60) | 54.5 | (38, 64) |
| Gender, n (%) | | | | |
| Female | 2 | (50%) | 2 | (29%) |
| Male | 2 | (50%) | 5 | (71%) |
| Race, n (%) | | | | |
| Caucasian | 4 | (100%) | 7 | (100%) |
| Ethnicity, n (%) | | | | |
| Not Hispanic or Latino | 4 | (100%) | 7 | (100%) |
| Karnofsky Performance | | | | |
| Score, n (%) | | | | |
| 100% | 1 | (25%) | 1 | (13%) |
| 90% | 2 | (50%) | 2 | (29%) |
| 80% | 1 | (25%) | 2 | (29%) |
| 70% | 0 | (0%) | 2 | (29%) |
| 60% | 0 | (—%) | 0 | (0%) |
| <60% | 0 | (—%) | 0 | (0%) |
| Number of Recurrences, n (%) | | | | |
| 1 | 1 | (25%) | 2 | (29%) |
| 2 | 2 | (50%) | 2 | (29%) |
| 3 or More | 1 | (25%) | 3 | (42%) |
| Days from GBM Diagnosis | | | | |
| to Enrollment | | | | |
| Median (Min, Max) | 1545 | (417, 5055) | 335 | (187, 991) |
| Days from Last Radiotherapy | | | | |
| to Enrollment | | | | |
| Median (Min, Max) | 686 | (618, 770) | 288 | (69, 841) |
| Days from Last Temozolomide | | | | |
| Dose to Enrollment | | | | |
| Median (Min, Max) | 578 | (509, 4942) | 143 | (1, 757) |

Summary of Safety Results:

There were no clinically significant changes on physical exams, including changes in vital signs and neurological exams, or in laboratory findings at any time point. A total of 55 TEAEs were reported. All patients reported at least one TEAE; none were related to the investigational device. One patient reported a serious adverse event, which was an infection of the ankle and not related to the investigational device. The most commonly reported TEAE was seizure. None of the patients stopped treatment or withdrew from the study due to TEAEs.

Summary of Clinical Utility:

A summary of clinical utility endpoints is shown below in Table 3. The median days on treatment was 134 days in the Voyager ulRFE® AIA signal alone group and 242 days in the Voyager ulRFE® AIA signal combined with BSC group. The longest treatment duration occurred in a patient in the Voyager ulRFE® AIA signal combined with BSC group, with treatment ongoing after 1000 days. The most frequently documented response by investigators was stable disease. These data suggest that the Voyager impacts survival.

TABLE 3
Clinical Utility Endpoints.

| | Treatment Arms | | | |
|---|---|---|---|---|
| | Voyager alone | | Voyager + BSC | |
| Endpoint | (N = 4) | | (N = 7) | |
| Days on Treatment | | | | |
| Median (Min, Max) | 134 | (27, 222) | 242 | (29, >1000) |
| Progression Free Survival (PFS) | | | | |
| Median (weeks) | 10 | | 16 | |
| PFS-6 | | | | |
| n (%) | 0 | (0%) | 3 | (43%) |
| Overall Survival (OS) | | | | |
| Median (months) | 16 | | 11 | |
| OS-6 | | | | |
| n (%) | 4 | (100%) | 7 | (100%) |
| OS-12 | | | | |
| n (%) | 2 | (50%) | 3 | (43%) |
| OS-18 | | | | |
| n (%) | 2 | (50%) | 2 | (29%) |
| OS-24 | | | | |
| n (%) | 2 | (50%) | 2 | (29%) |
| OS-26 | | | | |
| n (%) | 0 | (0%) | 1 | (14%) |

| | | |
|---|---|---|
| Tumor Response after 2 months | | |
| (by Investigator), n | | |
| Disease Controlled | 2 | 5 |
| Complete Response (CR) | 0 | 0 |
| Partial Response (PR) | 1 | 1 |
| Stable Disease (SD) | 1 | 4 |
| Progressive Disease (PD) | 2 | 2 |
| Deceased | 0 | 0 |

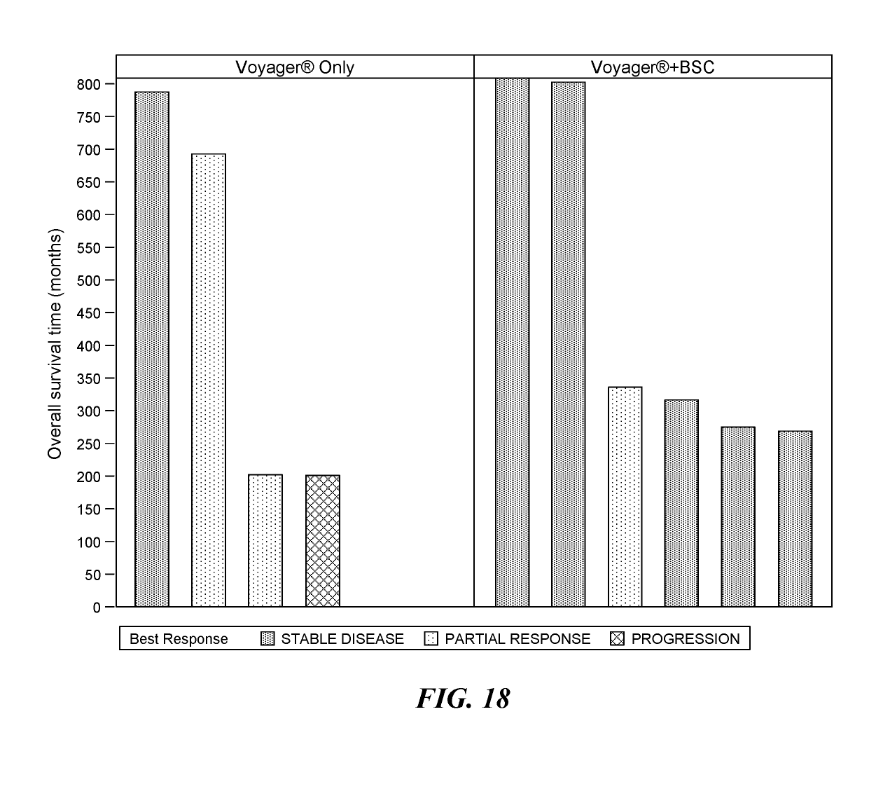

FIG. 18 shows the relationship between survival and tumor response. FIG. 18 indicates that overall, 7 (64%) subjects had their rGBM disease controlled, and these patients survived longer than those who progressed on study.

Overall, treatment with the Voyager system was safe as no device-related serious adverse events were reported. Of the 55 TEAEs reported during the study, none were related to the investigational device. The deaths that occurred on study were expected outcomes of recurrent GBM and not associated with use of the investigational device.

Data from the first 11 patients enrolled and treated with the Voyager were obtained. The median progression-free survival was 10 weeks in the monotherapy group and 16 weeks in the combination therapy group, and the median overall survival was 16 months in the monotherapy group and 11 months in the combination therapy group. The best overall tumor response in most patients was disease control (i.e., stable disease or partial response) and no serious adverse events associated with the investigational therapy were reported. Overall the tumor response data and survival outcomes suggest that the Nativis Voyager® system is useful in treating adults with rGBM.

As discussed above, the Voyager system can be programmed to include one or more of several different cognates. Using the ulRFE® cognate A2HU, the Voyager therapy blocks activity and/or expression of CTLA-4 and PD-1. The objective of this study was to assess whether the Voyager ulRFE® therapy was a safe and feasible treatment for rGBM.

The A2HU arm of this study was a single-site, prospective, open-label study conducted in Australia intended to assess the safety and feasibility of the Voyager system as a treatment for rGBM in patients following standard-of-care chemotherapy and radiotherapy. Two distinct ulRFE® cognates were studied. The first cohort of patients received treatment with A1A, a ulRFE® cognate derived from paclitaxel, and the second cohort received treatment with A2HU, a ulRFE® cognate derived from the siRNA targeting CTLA-4 and from the siRNA targeting PD-1. The treatment groups were not intended for comparison. Safety was assessed by incidence of any adverse events associated with the ulRFE® therapy. Tumor progression at each post-treatment visit was assessed via radiological response by local investigators. Patients were followed at least every 8 weeks during the first 6 months and every 4 months thereafter.

Safety and Clinical Utility Measurements:

Safety was assessed by incidence and evaluation of any adverse events associated with the investigational therapy, abnormal laboratory findings, and abnormal neurological findings. Clinical utility was assessed by tumor response after 2 months, PFS at 6 months, median PFS, OS at 6 and 12 months, and median OS. The radiological response of the tumor was assessed by MRI studies according to RANO or iRANO criteria. Patients in the A1A arm were assessed for PFS using the RANO criteria, while patients in the A2HU arm were assessed using the iRANO criteria. All patients had their tumor measurements recorded at baseline and at the time of each MRI scan. The dose and type of contrast agent was held constant from scan to scan for each patient.

Statistical Analysis:

The A1A and A2HU treatment groups were analyzed separately. The following analysis populations were defined:

(1) Safety Population: The safety population included all patients that received at least one day of treatment with the investigational device; and

(2) Treated Population: The treated population included all patients who received at least one month of treatment with the investigational device.

The data analyses were conducted using SAS® Software, version 9.4 or later. Baseline and demographic characteristics of the safety population were summarized. Continuous variables (age, baseline height) were summarized via mean, standard deviation, median, range, and number of non-missing responses. Categorical variables (gender, race, ethnicity, and KPS) were summarized via counts and percentages.

Adverse events (AEs) were graded according to the NCI Common Terminology Criteria for Adverse Event Version 3.0 (CTCAE V3.0) and were also coded using the Medical Dictionary for Regulatory Activities (MedDRA®). Treatment-emergent AEs (TEAEs), defined as any AE that occurred after a subject received the assigned study treatment, were summarized by the number and proportion of subjects reporting at least one occurrence of the AE. Frequencies of each TEAE were summarized by MedDRA® preferred term within system organ class (SOC), by severity grade, and relation to study device. Treatment emergent serious adverse events (TESAEs) were tabulated by MedDRA® preferred term within SOC.

Clinical laboratory tests were performed at pre-study (baseline) and at all visits. For each panel (hematology, biochemistry, coagulation) the study results were summarized in shift tables from baseline using the categories Normal, Abnormal (Not Clinically Significant), and Abnormal (Clinically Significant). All clinically significant abnormal findings were reported as AEs.

Physical exams, including vital signs and neurological exams, were performed at pre-study (baseline) and at all patient visits. Physical exam shift tables were constructed to summarize the changes in each body system from baseline for each assessed cycle.

Tumor response was assessed by the RANO or iRANO criteria, as appropriate to the treatment group, at each post-treatment visit. Subjects with unknown status for tumor response at the time point were excluded from the analysis.

For the median survival endpoints—i.e., OS (in months) and PFS (in weeks) —patients were followed until death. The start of the efficacy period for all analyses in this study was date of treatment initiation, Day 1. OS was assessed using death as the endpoint. PFS was determined using RANO or iRANO criteria, as appropriate to the treatment group. A waterfall plot of survival for each treatment arm was produced, displaying survival time and tumor response after 2 months of treatment for each patient.

Results

Twenty-eight patients were screened, and 17 were enrolled and received at least one day of treatment with the investigational device. The patient disposition (safety population) is enumerated in Table 4.

TABLE 4
Patient Disposition (Safety Population).

| | N = 17 | |
|---|---|---|
| Off Treatment Reasons, n (%) | | |
| Completed Treatment Schedule | 0 | (0.0%) |
| Documented Disease Progression | 5 | (29.4%) |
| Treatment Related Toxicity | 0 | (0.0%) |
| Non-treatment Related Toxicity | 0 | (0.0%) |
| Patient Requested Early Discontinuation | 4 | (23.5%) |
| of Trial but Still Followed | | |
| Physician Requested Early Discontinuation | 0 | (0.0%) |
| of Trial for Reasons Not Related to Toxicity | | |
| Death | 3 | (17.6%) |
| Non-Compliance | 0 | (0.0%) |
| Other | 4 | (23.5%) |
| Off Study Reasons, n (%) | | |
| Completed Treatment Schedule | 0 | (0.0%) |
| Documented Disease Progression | 0 | (0.0%) |
| Patient Requested Early Discontinuation | 0 | (0.0%) |
| of Trial but Still Followed | | |
| Lost to Follow Up | 0 | (0.0%) |
| Death | 16 | (94.1%) |
| Other | 0 | (0.0%) |

The patient demographics and other baseline characteristics are enumerated below in Table 5.

TABLE 5
Demographics and Baseline Characteristics (Safety Population).

| | Treatment Arms | | | |
|---|---|---|---|---|
| | A1A | | A2HU | |
| Characteristic | (N = 6) | | (N = 11) | |
| Age (years), n | | | | |
| Mean (SD) | 62.83 | (5.78) | 53.09 | (11.85) |
| Median (Min, Max) | 62.5 | (55, 70) | 55 | (37, 68) |
| Gender, n (%) | | | | |
| Female | 4 | (66.7%) | 5 | (45.5%) |
| Male | 2 | (33.3%) | 6 | (54.5%) |
| Race, n (%) | | | | |
| White | 6 | (100.0%) | 11 | (100.0%) |
| Ethnicity, n (%) | | | | |
| Not Hispanic or Latino | 6 | (100.0%) | 11 | (100.0%) |
| Karnofsky Performance | | | | |
| Score, n (%) | | | | |
| 100% | 3 | (50.0%) | 1 | (9.1%) |
| 90% | 1 | (16.7%) | 3 | (27.3%) |
| 80% | 0 | (0.0%) | 2 | (18.2%) |
| 70% | 2 | (33.3%) | 3 | (27.3%) |
| 60% | 0 | (0.0%) | 2 | (18.2%) |
| <60% | 0 | (0.0%) | 0 | (0.0%) |
| Number of | | | | |
| Recurrences, n (%) | | | | |
| 1 | 1 | (16.7%) | 5 | (45.5%) |
| 2 | 5 | (83.3%) | 6 | (54.5%) |
| 3 or More | 0 | (0.0%) | 0 | (0.0%) |
| Days from GBM Diagnosis | | | | |
| to Enrollment | | | | |
| Median (Min, Max) | 493 | (189, 1098) | 467 | (235, 2872) |
| Days from Last Radiotherapy | | | | |
| to Enrollment | | | | |
| Median (Min, Max) | 431 | (111, 1022) | 386 | (264, 1364) |
| Days from Last | | | | |
| Temozolomide | | | | |
| Dose to Enrollment | | | | |
| Median (Min, Max) | 267 | (39, 1022) | 386 | (82, 1364) |

Summary of Safety:

There were no clinically significant changes on physical exams (including changes in vital signs and neurological exams) or in laboratory findings (data not shown). A TEAE summary for the study is presented in Table 6. The majority of patients who reported at least one TEAE had a relationship of unlikely or unrelated to Voyager therapy. Only one patient (treated with cognate A2HU) reported a TEAE that was possibly related to Voyager therapy. The TEAE was an unresolved increase in headaches with no action taken.

TABLE 6
Treatment Emergent Adverse Event Summary (Safety Population).

| | A1A | | A2HU | |
|---|---|---|---|---|
| | (N = 6) | | (N = 11) | |
| Subjects with at Least One Adverse Event | 6 | (100.0%) | 10 | (90.9%) |
| Highest AE Severity Grade | | | | |
| Grade 1 Mild [n(%)] | 0 | (0.0%) | 0 | (0.0%) |
| Grade 2 Moderate [n(%)] | 1 | (16.7%) | 5 | (45.5%) |
| Grade 3 Severe or Medically | 5 | (83.3%) | 3 | (27.3%) |
| Significant [n(%)] | | | | |
| Grade 4 Life-threatening or | 0 | (0.0%) | 0 | (0.0%) |
| Disabling [n(%)] | | | | |
| Grade 5 Death [n(%)] | 0 | (0.0%) | 2 | (18.2%) |
| Strongest Relationship of AE | | | | |
| Not Related [n(%)] | 4 | (66.7%) | 1 | (9.1%) |
| Unlikely [n(%)] | 2 | (33.3%) | 8 | (72.7%) |
| Possibly [n(%)] | 0 | (0.0%) | 1 | (9.1%) |
| Probably [n(%)] | 0 | (0.0%) | 0 | (0.0%) |
| Definitely [n(%)] | 0 | (0.0%) | 0 | (0.0%) |
| Subjects Experiencing at Least | 4 | (66.7%) | 8 | (72.7%) |
| One Serious Adverse Event | | | | |
| Deaths | 1 | (16.7%) | 2 | (18.2%) |

Adverse events were coded with MedDRA Coding Dictionary Version 19.1. The TEAEs that occurred most frequently by MedDRA preferred term (>20% frequency within an individual arm) are summarized by preferred term and system organ class in Table 7.

In the A2HU arm, the most frequently reported TEAE was headache, which was reported by 6 patients (54.5%). The next most commonly reported TEAE was seizure, which was reported by 5 patients (45.5%). The following TEAEs were reported by 4 patients: amnesia, aphasia, and confusional state.

In the A1A arm, the most frequently reported TEAEs were amnesia and aphasia, each of which was reported by 3 patients (50%). The following TEAEs were reported in 2 patients: hemiparesis and nausea.

TABLE 7
Number and Percentage of Subjects with Adverse Events for Most
Frequently Reported Preferred Terms (Safety Population).

| | A1A | | A2HU | |
|---|---|---|---|---|
| | (N = 8) | | (N = 11) | |
| Total Number of Adverse Events | 74 | | 119 | |
| Subjects with at Least One Adverse Event | 8 | (100.0%) | 10 | (90.9%) |
| GASTROINTESTINAL DISORDERS | 3 | (50.0%) | 3 | (27.3%) |
| NAUSEA | 2 | (33.3%) | 0 | (0.0%) |
| VOMITING | 0 | (0.0%) | 3 | (27.3%) |
| INJURY, POISONING AND | 3 | (37.5%) | 3 | (27.3%) |
| PROCEDURAL COMPLICATIONS | | | | |
| FALL | 1 | (16.7%) | 3 | (27.3%) |
| NERVOUS SYSTEM DISORDERS | 8 | (100.0%) | 9 | (81.8%) |
| AMNESIA | 3 | (50.0%) | 4 | (36.4%) |
| APHASIA | 3 | (50.0%) | 4 | (36.4%) |
| BALANCE DISORDER | 0 | (0.0%) | 3 | (27.3%) |
| DISTURBANCE IN ATTENTION | 0 | (0.0%) | 3 | (27.3%) |
| HEADACHE | 1 | (16.7%) | 6 | (54.5%) |
| HEMIPARESIS | 2 | (33.3%) | 3 | (27.3%) |
| SEIZURE | 0 | (0.0%) | 5 | (45.5%) |
| PSYCHIATRIC DISORDERS | 3 | (37.5%) | 7 | (63.6%) |
| CONFUSIONAL STATE | 0 | (0.0%) | 4 | (36.4%) |

Summary of Clinical Utility:

A summary of clinical utility endpoints is shown below in Table 8. The median days on treatment was 168 days in the A1A arm and 202 days in the A2HU arm. The longest treatment duration occurred in a patient in the A1A arm, with treatment spanning 342 days.

The most frequently documented response was stable disease, which was documented in 4 patients in the A1A arm and 6 in the A2HU arm. Two patients in the A2HU arm achieved a partial response, and none of the patients achieved a complete response. Overall, 12 (80%) subjects had their disease controlled.

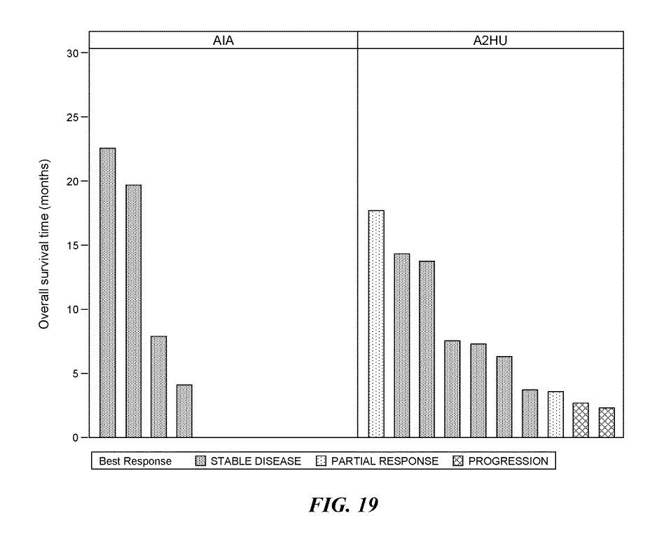

Time to progression and time to death are also summarized in Table 8. None of the subjects were censored for the analysis. An overall survival waterfall plot is provided in FIG. 19 to illustrate the relationship between the tumor response after 2 months and the overall survival time (in months) in each patient. One patient in the A1A arm did not have a tumor assessment at 2 months and is therefore not represented in the FIG. 19.

TABLE 8
Summary of Clinical Utility (Treated Population).

| | Treatment Arms | | | |
|---|---|---|---|---|
| | A1A | | A2HU | |
| Endpoint | (N = 5) | | (N = 10) | |
| Days on Treatment | | | | |
| Median (Min, Max) | 168 | (34, 342) | 202 | (54, 266) |
| Progression Free Survival (PFS) | | | | |
| Median (weeks) | 16.14 | | 11.93 | |
| PFS-6 | | | | |
| n (%) | 1 | (20%) | 3 | (30%) |
| Overall Survival (OS) | | | | |
| Median (months) | 8.04 | | 6.89 | |
| OS-6 | | | | |
| n (%) | 3 | (60%) | 6 | (60%) |
| OS-12 | | | | |
| n (%) | 2 | (40%) | 3 | (30%) |
| Tumor Response after 2 months | | | | |
| (by Investigator), n | | | | |

| | | |
|---|---|---|
| Disease Controlled | 4 | 8 |
| Complete Response (CR) | 0 | 0 |
| Partial Response (PR) | 0 | 2 |
| Stable Disease (SD) | 4 | 6 |
| Progressive Disease (PD) | 0 | 2 |
| Deceased | 0 | 0 |
| Unknown/unreported | 1 | 0 |

Overall, treatment with the Voyager system was safe. No device-related serious adverse events were reported. Of the 193 TEAEs observed during the study, only 1 event was reported as possibly related to the device. All other TEAEs were either not related or unlikely related to the device. The deaths that occurred on study were expected outcomes of recurrent GBM and not associated with use of the device.

Five patients were enrolled and treated per protocol using the Voyager A1A therapy, and 10 patients were enrolled and treated per protocol using the Voyager A2HU therapy. Median overall survival was 8.04 months in the A1A arm and 6.89 months in the A2HU arm. No serious adverse events associated with the investigational therapy were reported. Two subjects in the A2HU arm achieved a partial response, and 10 subjects overall (4 subjects in A1A and 6 subjects in A2HU) achieved stable disease. Median times to progression were 16.14 weeks for patients in the A1A arm and 11.93 weeks for patients in the A2HU arm.

There were no treatment-emergent serious adverse events reported that were related to the study device. No clinically relevant trends were noted in clinical laboratory parameters, vital signs, or physical exams. Treatment with Voyager was generally well tolerated, with median days on treatment of 168 days (24 weeks) in patients in the A1A arm and 202 days (approximately 29 weeks) in patients in the A2HU arm. The majority of patients achieved disease control, with a best overall response of either stable disease or partial response.

These data suggest that the Nativis Voyager® system is safe and feasible (i.e., has clinical utility) for the treatment of rGBM.

### Example 4. Nativis Voyager® System in Patients with Newly Diagnosed rGBM—Prophetic

Based on the feasibility and clinical utility Nativis Voyager® system in the treatment of rGBM as described in Examples 2 and 3 above, a feasibility study in patients with newly diagnosed GBM will be undertaken to determine if the Voyager ulRFE® therapy is a safe and feasible treatment for newly diagnosed GBM. This will be a prospective, open-label, multi-center trial. In this trial, adults newly diagnosed with GBM, following maximal tumor debulking, are eligible for enrollment.

Objective.

The objective of the study is to assess if the Voyager ulRFE® therapy is a safe and feasible treatment for newly diagnosed GBM when combined with standard of care (i.e., focal radiotherapy plus temozolomide).

Methods.

Patients will receive continual therapy with the Nativis Voyager® system, concurrently with radiotherapy and temozolomide. Upon progression, investigators can choose to either maintain patients on study with the Nativis Voyager® and to add second-line therapy.

Outcomes.

The primary outcome measure is safety, which will be assessed by the incidence and evaluation of any serious adverse events associated with the Nativis Voyager® system through follow-up. The secondary outcome measure is clinical utility, which will be assessed by progression free survival and overall survival.

The system described herein transduces a specific ulRFE® of a molecule, or cognate, to effect a specific charge pathway and may be configured to deliver the effect of chemical, biochemical or biologic treatment to humans, mammals, and/or animals and treat an adverse health condition or produce another biological effect, without the use of drugs or chemicals, alternative therapies, etc. For example, the system can transduce RNA sequence ulRFE® to regulate metabolic pathways and protein production, both up regulation and down regulation.

The system provides numerous other benefits. The system is scalable to provide treatment to a variety of human, mammal, and/or animal regions or configurations. The coil, cable and connector are disposable, or the device as a whole with the controller, are preferably provided for a single treatment session, so that the device and coil are not to be reused, thereby preventing cross contamination, etc. The switch on the device permits an on-off nature of treatment so that it may be selectively switched on and off if needed.

Unless the context clearly requires otherwise, throughout the description and the claims, the words “comprise,” “comprising,” and the like are to be construed in an inclusive sense, as opposed to an exclusive or exhaustive sense; that is to say, in the sense of “including, but not limited to.” The word “coupled,” as generally used herein, refers to two or more elements that may be either directly connected, or connected by way of one or more intermediate elements. Additionally, the words “herein,” “above,” “below,” and words of similar import, when used in this application, shall refer to this application as a whole and not to any particular portions of this application. Where the context permits, words in the above Detailed Description using the singular or plural number may also include the plural or singular number respectively. The word “or” in reference to a list of two or more items, that word covers all of the following interpretations of the word: any of the items in the list, all of the items in the list, and any combination of the items in the list.

The above detailed description of embodiments of the invention is not intended to be exhaustive or to limit the invention to the precise form disclosed above. While specific embodiments of, and examples for, the invention are described above for illustrative purposes, various equivalent modifications are possible within the scope of the invention, as those skilled in the relevant art will recognize. For example, while processes or blocks are presented in a given order, alternative embodiments may perform routines having steps, or employ systems having blocks, in a different order, and some processes or blocks may be deleted, moved, added, subdivided, combined, and/or modified. Each of these processes or blocks may be implemented in a variety of different ways. Also, while processes or blocks are at times shown as being performed in series, these processes or blocks may instead be performed in parallel or may be performed at different times.

Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with some embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

The teachings of the invention provided herein can be applied to other systems, not necessarily the system described above. The elements and acts of the various embodiments described above can be combined to provide further embodiments.

All of the above patents and applications and other references, including any that may be listed in accompanying filing papers, are incorporated herein by reference. Aspects of the invention can be modified, if necessary, to employ the systems, functions, and concepts of the various references described above to provide yet further embodiments of the invention.

These and other changes can be made to the invention in light of the above Detailed Description. While the above description details some embodiments of the invention and describes the best mode contemplated, no matter how detailed the above appears in text, the invention can be practiced in many ways. Details of the signal processing system may vary considerably in its implementation details, while still being encompassed by the invention disclosed herein. As noted above, particular terminology used when describing some features or aspects of the invention should not be taken to imply that the terminology is being redefined herein to be restricted to any specific characteristics, features, or aspects of the invention with which that terminology is associated. In general, the terms used in the following claims should not be construed to limit the invention to the specific embodiments disclosed in the specification, unless the above Detailed Description section explicitly defines such terms. Accordingly, the actual scope of the invention encompasses not only the disclosed embodiments, but also all equivalent ways of practicing or implementing the invention under the claims.

### REFERENCES

1. Robitaille P M, Kangarlu A, Abduljalil A M. RF penetration in ultra-high field MRI: challenges in visualizing details within the center of the human brain. J Comput Assist Tomogr. 1999; 23(6):845-9. Epub 1999/12/10. PubMed PMID: 10589557.
2. Roschmann P. Radiofrequency penetration and absorption in the human body: limitations to high-field whole-body nuclear magnetic resonance imaging. Med Phys. 1987; 14(6):922-31. Epub 1987/11/01. PubMed PMID: 3696080.
3. Bottomley P A, Andrew E R. RF magnetic field penetration, phase shift and power dissipation in biological tissue: implications for NMR imaging. Phys Med Biol. 1978; 23(4):630-43. Epub 1978/07/01. PubMed PMID: 704667.
4. Wang et al., Natural taxanes: development since 1828, Chem. Rev. 111(12): 7652-7709 (2011).
5. Shi et al., New minor taxane derivatives from the needles of Taxus Canadensis, J. Nat. Prod. 66 (11): 1480-1485 (2003).

1. A method of treating glioblastoma or recurrent glioblastoma in a subject comprising administering to the subject one or more ulRFE® signals.

2. The method of claim 1, wherein the one or more ulRFE® signals are administered using the Nativis Voyager® system.

3. The method of claim 1, further comprising administering to the subject a chemotherapy or an anti-angiogenic therapy or other cancer therapy.

4. The method of claim 3, wherein the anti-angiogenic therapy is Avastin.

5. The method of claim 1, wherein the one or more ulRFE® signals further comprises two signals.

6. The method of claim 1, wherein the one or more ulRFE® signals further comprises three signals.

7. The method of claim 1, wherein the one or more ulRFE® signals further comprises three or more signals.

8. A method of treating newly diagnosed glioblastoma in a subject comprising administering to the subject one or more ulRFE® signals.

9. The method of claim 8, wherein the subject is also being treated with a chemotherapy or radiotherapy.

10. The method of claim 9, wherein the treatment with chemotherapy further comprises administering temozolomide to the subject.

11. The method of claim 10, wherein the temozolomide is administered to the subject before, during, and/or after administration of the one or more ulRFE® signals.

12. The method of claim 8, wherein the subject does not exhibit any serious adverse events attributable to the ulRFE® signal after administration of the one or more ulRFE® signals.

13. The method of claim 8, wherein each of the one or more ulRFE® signals are derived from a mitotic inhibitor or an siRNA molecule.

14. The method of claim 13, wherein the mitotic inhibitor is a taxane-derivative.

15. The method of claim 14, wherein the taxane-derivative is taxol or paclitaxel.

16. The method of claim 13, wherein the siRNA molecule is an siRNA molecule targeting CTLA-4 or PD-1.

17.-26. (canceled)
